(19) United States
(12) Patent Application Publication
(10) Pub. No.: US 2024/0262786 A1
(43) Pub. Date: Aug. 8, 2024

(54) METHOD FOR OBTAINING BIO-SOURCED SUSBTITUTED ALKYL(METH)ACRYLAMIDE

(71) Applicant: SPCM SA, ANDREZIEUX- BOUTHEON (FR)

(72) Inventors: Cédrick FAVERO, ANDREZIEUX BOUTHEON (FR); Johann KIEFFER, ANDREZIEUX BOUTHEON (FR)

(21) Appl. No.: 18/562,742

(22) PCT Filed: Jul. 8, 2022
(86) PCT No.: PCT/EP2022/069145

§ 371 Date: Nov. 20, 2023

(30) Foreign Application Priority Data
Jul. 9, 2021 (FR) FR2107500

(51) Int. Cl. C07C 231/02; C02F 1/56; C02F 11/147; C02F 103/12; C04B 24/16; C04B 103/46; C07C 233/09; C07C 233/38; C08F 220/56; C09K 8/12; C09K 8/487; C09K 8/584; C09K 8/588; C09K 8/60; C09K 8/68; C12P 7/40; E21B 33/14; E21B 43/16; E21B 43/26

(52) U.S. Cl. CPC C07C 231/02; C02F 1/56; C02F 11/147; C04B 24/163; C07C 233/09; C07C 233/38; C08F 220/56; C09K 8/12; C09K 8/487; C09K 8/584; C09K 8/588; C09K 8/604; C09K 8/68; C12P 7/40; E21B 33/14; E21B 43/16; E21B 43/26; C02F 2103/12; C04B 2103/46; C08F 2800/20

(57) ABSTRACT

A method for obtaining substituted alkyl(meth)acrylamide includes reaction between (meth)acrylic acid or an ester thereof, and a primary or a secondary alkylamine. At least one of the two is at least partially renewable and non-fossil. The substituted alkyl(meth)acrylamide can have a bio-sourced carbon content of between 5 wt % and 100 wt % relative to the total carbon weight in the substituted alkyl(meth)acrylamide. The bio-sourced carbon content can be measured according to the standard ASTM D6866-21 Method B.

### FIELD OF THE INVENTION

The present invention relates to a method for obtaining bio-sourced substituted alkyl(meth)acrylamide comprising the reaction between (meth)acrylic acid or one of the esters thereof on the one hand, and a primary alkylamine on the other hand, one of the two, preferentially both, being at least partially renewable and non-fossil. The invention relates to the bio-sourced substituted alkyl(meth)acrylamide monomer as well as a bio-sourced polymer obtained from at least one bio-sourced substituted alkyl(meth)acrylamide monomer according to the invention. Lastly, the invention relates to the use of the invention's bio-sourced polymers in various technical fields.

### PRIOR ART

Ethylenically unsaturated monomers, such as substituted alkyl(meth)acrylamides are widely used in manufacturing water-soluble or water-swellable polymers.

Substituted alkyl(meth)acrylamides are generally obtained according to the following reaction pathway.

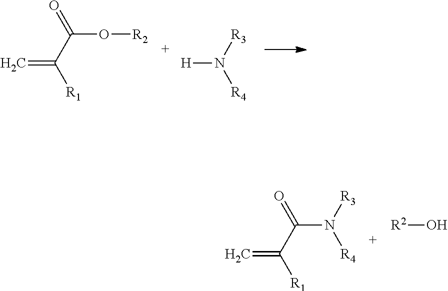

Where R₁═H or CH₃, R₂═H, alkyl chain containing 1 to 4 carbon atoms, or Cl, R₃═H, or alkyl chain containing 1 to 8 carbon atoms, R₄=alkyl chain containing 1 to 8 carbon atoms, or alkylamine containing 1 to 4 carbon atoms (dimethylaminopropyl), or alkanol amine containing 1 to 4 carbon atoms. Aliphatic chains can be linear, branched or ringed. Generally, they are linear.

R₃ and R₄ can form a heterocyclic ring comprising 4 to 6 carbon atoms. This is particularly the case for morpholine.

Several variants exist depending on the nature of R₂.

In the case where R₂ is a halogen, generally a chlorine, the substituted alkyl(meth)acrylamides are synthesized by the Schotten Baumann reaction where acryloyl chloride is put in contact with an alkylamine and a base. The synthesis can be carried out in aqueous or solvent phase. The base is used to neutralize the hydrochloric acid that is generated as a by-product. This base can be soda, potash, sodium carbonate or an organic base like triethylamine. Meanwhile, acryloyl chloride is generated from the chlorination of acrylic acid, where the chlorinating agent can be phosgene (carbon oxychloride), thionyl chloride, phosphorus trichloride or phosphorus pentachloride. The following documents describe this process: EP 0 115 703, U.S. Pat. No. 5,324,765 and JP-2013-95666.

It is known that the direct reaction between an alkylamine with acrylic acid, or an ester thereof, leads to the formation of Michael adduct. To counteract this drawback, an alternative synthetic process has been developed according to the following reaction pathway:

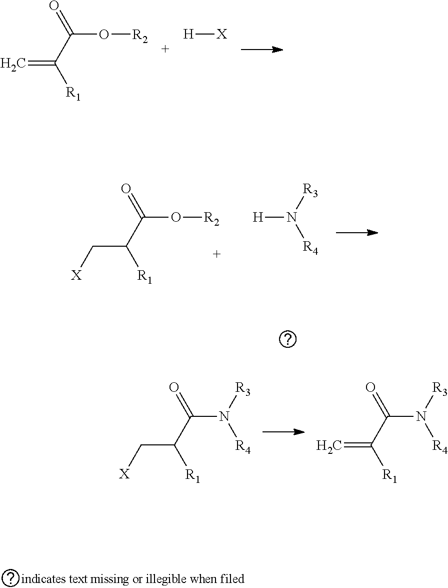

R₁, R₂, R₃ and R₄ are identical to the previous description. X is advantageously an alkoxy or an aliphatic amine, more preferentially X is CH₃O— or HNR₃R₄, more preferentially HNR₃R₄.

The synthesis proceeds in three steps, with first the reaction between an acrylic ester and a protecting agent of generic formula HX to form an intermediate. This intermediate is then reacted with an alkylamine to form a second intermediate. Lastly, this last intermediate undergoes a chemical reaction to break the bond between the terminal carbon and the protecting agent X, in order to generate a double bond. This step is generally called the retro Michael reaction.

The protecting agents can be of different types, for example document JP 2015-209419 describes the use of an alkylamine. This alkylamine is generally of the same type as the amine used to synthesize intermediate 2. Document U.S. Pat. No. 4,237,067 describes the use of a hydroxide as a protective agent. Document EP 1 357 05 describes the use of an alcohol as a protecting agent.

In all these documents, the step known as retro Michael, which generates the double bond, must be carried out at very high temperature according to a pyrolysis method. The high temperature used generates a set of by-products and induces polymerizations of the reaction medium.

Additionally, the conversions are generally not complete, and the protective agent obtained from the pyrolysis reacts again with the alkyl(meth)acrylamide generated during the pyrolysis, to re-synthesize the second intermediate. To counteract these drawbacks, the various previous documents also describe the use of catalysts to lower the temperatures required for the pyrolysis step, or distillation purification systems to separate the Michael adducts with the product of interest. Substituted alkyl(meth)acrylamides being by nature reactive monomers, a fraction of the mixture to be separated polymerizes, which results in a loss of yield, in the generation of a polymer which must be destroyed and in a loss of productivity of the production unit induced by the stoppage of the distillation column for cleaning purposes.

Acrylic acid ester is obtained by esterification between acrylic acid and an alcohol, generally catalyzed with an acid such as para-toluene sulfonic acid, Nafion resin, sulfuric acid, or methane sulfonic acid as in Document WO 2015/015100 for example.

There are many patent documents describing how to obtain bio-sourced acrylic acid, such as Document US 2010/0168471, for example, which describes the transformation of glycerol into acrylic acid, Document WO 2012/074818 which describes the fermentation of biomass to obtain a 3-hydroxypropionic acid intermediate, the latter being a chemical precursor of acrylic acid.

Alkylamine is obtained by reaction between an alcohol and ammonia. For example, in the case of dimethylamine, methanol is reacted with ammonia, as described in document U.S. Pat. No. 4,582,936.

Methanol is obtained by steam reformation of methane, or via partial oxidation of methane. In the case of diethylamine, ethanol is reacted with ammonia, as described in document U.S. Pat. No. 4,314,084. Ethanol is obtained by direct hydration of ethylene. In the case of diisopropylamine, isopropanol is reacted with ammonia, as described in document CN107459465. Isopropanol is obtained by reduction of acetone with hydrogen, or by direct hydration of propylene.

In the case of morpholine, diethylene glycol is reacted with ammonia, as described in document U.S. Pat. No. 4,739,051. Diethylene glycol is obtained from ethylene oxide, the latter being obtained by oxidation of ethylene. Fossil-based ethylene contains various impurities, which remain or are transformed in the method for producing ethylene oxide and thus producing morpholine.

In the particular case of N, N dimethylaminopropylamine, acrylonitrile is reacted with dimethylamine to obtain an intermediate dimethylaminopropionitrile which is then hydrogenated, as described in the U.S. Pat. No. 7,723,547.

Acrylonitrile is currently produced by an ammoxidation method, commonly known as the SOHIO method, by reaction between propylene and ammonia, as described in U.S. Pat. No. 2,904,580.

Propylene is a fossil-based olefin, and is currently produced by steam cracking of naphtha, itself derived from crude oil refining. More recently, and with the advent of shale gas production, various propane dehydrogenation methods to produce propylene have been described. Fossil-based propylene contains various impurities, which remain or are transformed by the ammoxidation method.

The problem the invention proposes to resolve is to provide a new and improved method for producing substituted alkyl(meth)acrylamide.

### SUMMARY OF THE INVENTION

Surprisingly, the applicant has observed that the use of (meth)acrylic acid or one of the esters thereof that is at least partially renewable and non-fossil, and/or an alkylamine that is at least partially renewable and non-fossil, in a method for obtaining substituted alkyl(meth)acrylamide allows to substantially improve the method and the quality of the monomer obtained, thereby improving the polymerization and the application performance of the polymers.

Without seeking to be bound by any particular theory, the Applicant raises the possibility that the different nature of the impurities between fossil-based and a renewable and non-fossil-based compound is the cause of these unexpected technical effects.

The Applicant has particularly observed this improvement where the (meth)acrylic acid used in the method or the (meth)acrylic acid used to obtain the corresponding (meth)acrylic acid ester, the latter being used in the method of the invention, is obtained according to a method comprising at least one enzymatic bioconversion step.

A first object of the invention is a method for obtaining substituted alkyl(meth)acrylamide comprising the reaction between (meth)acrylic acid or one of the esters thereof on the one hand, and a primary or a secondary alkylamine on the other hand, one of the two, preferentially both, being at least partially renewable and non-fossil. Preferably, the method comprises at least one step for obtaining (meth)acrylic acid by bioconversion in the presence of a biocatalyst comprising at least one enzyme.

The term “alkyl(meth)acrylamide” refers to alkylacrylamide and alkylmethacrylamide with a nitrogen atom that is monosubstituted or disubstituted.

Another object of the invention is a substituted bio-alkyl(meth)acrylamide obtained by reaction between (meth)acrylic acid or one of the esters thereof on the one hand, and a primary or a secondary alkylamine on the other hand, one of the two, preferentially both, being at least partially renewable and non-fossil.

Another object of the invention is a polymer obtained by polymerization of at least one substituted bio-alkyl(meth)acrylamide obtained by the method according to the invention, or obtained by polymerization of at least one substituted bio-alkyl(meth)acrylamide according to the invention. A further object of the invention is using the polymer according to the invention in various technical fields.

With the present invention, it is possible to achieve environmental objectives inherent in new technical innovations. In the present case, the use of renewable and non-fossil raw material allows to substantially optimize the method. It also allows to obtain polymerizable bio-monomers which deliver unexpectedly improved performances.

The Applicant observed that secondary alkylamine of renewable and non-fossil origin has a higher conversion rate of (meth)acrylic acid or one of its esters, than secondary alkylamine of fossil origin.

It also involves fewer impurities.

The Applicant also observed that polymers obtained with bio-sourced monomers are more easily biodegradable than polymers that do not contain bio-based monomers.

The Applicant also observed that polymers obtained with bio-sourced monomers exhibit an improved control of cement fluid loss as compared to polymers that do not contain bio-based monomers.

The Applicant also observed that polymers obtained with bio-sourced monomers exhibit greater solid particles retention properties than polymers that do not contain bio-based monomers.

The Applicant also observed that polymers obtained with bio-sourced monomers exhibit greater flocculant properties than polymers that do not contain bio-based monomers.

### DETAILED DESCRIPTION OF THE INVENTION

In the context of the invention, the terms “renewable and non-fossil” are used to designate the origin of a chemical compound derived from biomass or from synthesis gas (syngas), i.e. resulting from one or more chemical transformations carried out on one or more natural and non-fossil raw materials. The terms “bio-sourced” or “bio-resourced” can also be used to characterize the renewable and non-fossil origin of a chemical compound. The renewable and non-fossil origin of a compound includes renewable and non-fossil raw materials stemming from the circular economy, which have been previously recycled, once or several times, in a biomass material recycling process, such as materials from polymer depolymerization or pyrolysis oil processing.

According to the invention, the “at least partially renewable and non-fossil” quality of a compound means a bio-sourced carbon content preferably between 5 wt % and 100 wt % relative to the total carbon weight of said compound.

In the context of the invention, the ASTM D6866-21 standard Method B is used to characterize the bio-sourced nature of a chemical compound and to determine the bio-sourced carbon content of said compound. The value is expressed as a weight percentage (wt %) of bio-sourced carbon relative to the total carbon weight in said compound.

The ASTM D6866-21 standard is a test method that teaches how to experimentally measure the bio-sourced carbon content of solids, liquids and gaseous samples by radiocarbon analysis.

This standard primarily uses Accelerator Mass Spectrometry (AMS) technology. This technique is used to naturally measure the radionuclides present in a sample, wherein the atoms are ionized, then accelerated to high energies, then separated, and individually counted in Faraday cups. This high-energy separation is extremely effective at filtering out isobaric interference, so that AMS is able to accurately measure abundances of carbon-14 relative to carbon-12 (14C/12C) to an accuracy of 1.10⁻¹⁵.

The ASTM D6866-21 standard Method B uses AMS and IRMS (Isotope Ratio Mass Spectroscopy). The test method allows to directly differentiate contemporary carbon-based carbon atoms from fossil-based carbon atoms. A measure of the carbon-14 to carbon-12 or carbon-14 to carbon-13 content of a product is determined against a modern carbon-based reference material accepted by the radiocarbon dating community such as the NIST's Standard Reference Material (SRM) 4990C (oxalic acid).

The sample preparation method is described in the standard and does not require any special comment as it is a commonly used procedure.

Analysis, interpretation and reporting of results are described below. Isotope ratios of carbon-14 to carbon-12 content or carbon-14 to carbon-13 content are measured using AMS. Isotope ratios of carbon-14 to carbon-12 content or carbon-14 to carbon-13 content are determined relative to a standard traceable via the NIST SRM 4990C modern reference standard. The “fraction of modern” (fM) represents the amount of carbon-14 in the tested product relative to the modern standard. It is most often referred to as percent modern carbon (pMC), the percentage equivalent to fM (e.g. fM 1=100 pMC).

All pMC values obtained from radiocarbon analyses must be corrected for isotopic fractionation using a given stable isotope. The correction should be made using the carbon-14 to carbon-13 values determined directly using the AMS where possible. If this is not possible, the correction should be made using the delta 13C (δ13C) measured by IRMS, CRDS (Cavity Ring Down Spectroscopy) or any other equivalent technology that can provide accuracy to within plus or minus 0.3 per thousand.

“Zero pMC” represents the total absence of measurable 14C in a material above the background signals, thus indicating a fossil (e.g. petroleum-based) carbon source. A value of 100 pMC indicates a fully “modern” carbon source. A pMC value between 0 and 100 indicates a proportion of carbon derived from a fossil source relative to a “modern” source.

The pMC may be higher than 100% due to the persistent, but diminishing, effects of 14C injection into the atmosphere caused by atmospheric nuclear testing programmes. The pMC values need to be adjusted by an atmospheric correction factor (REF) to obtain the actual bio-sourced content of the sample.

The correction factor is based on the excess 14C activity in the atmosphere at the time of testing. A REF value of 102 pMC was determined for 2015 based on CO₂ measurements in the air in a rural area of the Netherlands (Lutjewad, Groningen). The first version of this standard (ASTM D6866-04) in 2004 had referenced a value of 107.5 pMC, while the later version ASTM D6866-(2010) had referenced a value of 105 pMC. These data points represent a drop of 0.5 pMC per year. Consequently, on 2 January of each year, the values in Table 1 below were used as REF value until 2019, reflecting the same decrease of 0.5 pMC per year. The REF values (pMC) for 2020 and 2021 have been determined to be 100.0 based on continuous measurements in the Netherlands (Lutjewad, Groningen) until 2019. References for reporting carbon isotope ratio data are provided below for 14C and 13C, respectively Roessler, N., Valenta, R. J., and van Cauter, S., “Time-resolved Liquid Scintillation Counting”, Liquid Scintillation Counting and Organic Scintillators, Ross, H., Noakes, J. E., and Spaulding, J. D., Eds., Lewis Publishers, Chelsea, M I, 1991, pp. 501-511. Allison, C. E., Francy, R. J., and Meijer, H. A. J., “Reference and Intercomparison Materials for Stable Isotopes of Light Elements”, International Atomic Energy Agency, Vienna, Austria, IAEATECHDOC-825, 1995.

The percentage of the bio-sourced carbon content is calculated by dividing pMC by REF and multiplying the result by 100. For example, [102 (pMC)/102 (REF)]×100=100% bio-sourced carbon. The results are indicated as a weight percentage (wt %) of bio-sourced carbon relative to the total carbon weight in said compound.

TABLE 1
Reference of percentage of modern carbon (pMC)

| REF year | pMC |
|---|---|
| 2015 | 102.0 |
| 2016 | 101.5 |
| 2017 | 101.0 |
| 2018 | 100.5 |
| 2019 | 100.0 |
| 2020 | 100.0 |
| 2021 | 100.0 |

In the context of the invention, the term “segregated” means a material stream that is distinctive and distinguishable from other material streams in a value chain (e.g. in a product manufacturing method), and thus considered to belong to a set of materials having an equivalent nature, such that the same origin of the material, or its manufacture according to the same standard or norm, can be tracked and guaranteed throughout this value chain.

For example, this may be the case of a chemist buying 100% bio-sourced (primary or secondary) alkylamine exclusively from a single supplier who guarantees the 100% bio-sourced origin of the alkylamine delivered, and said chemist processing this 100% bio-sourced alkylamine separately from other potential alkylamine sources to produce a chemical compound. If the chemical compound produced is made solely from said 100% bio-sourced alkylamine, then the chemical compound is 100% bio-sourced.

In the context of the invention, the term “non-segregated”, in contrast to the term “segregated”, is understood to mean a material stream that cannot be differentiated from other material streams in a value chain.

In order to better understand this notion of segregation, it is useful to recall some basics about the circular economy and its practical application in methods, especially chemical transformation.

According to the French Environment and Energy Management Agency (ADEME), the circular economy can be defined as an economic system of trade and production which, at all stages of the life cycle of products (goods and services), seeks to increase efficiency in the use of resources and to reduce the environmental impact while developing the well-being of individuals. In other words, it is an economic system devoted to efficiency and sustainability that minimizes waste by optimizing value generated by resources. It relies heavily on a variety of conservation and recycling practices in order to break away from the current more linear “take-make-dispose” approach.

In the field of chemistry, which is the science of transforming one substance into another, this translates into reusing material that has already been used to make a product. Theoretically, all chemicals can be isolated and therefore recycled separately from other chemicals. The reality, particularly in industry, is more complex and means that even when isolated, the compound cannot often be differentiated from the same compound originating from another source, thus complicating the traceability of the recycled material.

For this reason, various traceability models have been developed taking into account this industrial reality, thereby allowing users in the chemical industry to manage their material streams with full knowledge of the facts, and allowing end customers to understand and know in a simple way the origin of the materials used to produce an object or a commodity.

These models have been developed to build transparency and trust throughout the value chain. Ultimately, this allows end-users or customers to choose a more sustainable solution without having the ability themselves to control every aspect of the method, by knowing the proportion of a desired component (e.g. of a bio-sourced nature) in an object or commodity.

One such model is “segregation”, which we have defined earlier. Some known examples where this model applies are glass and some metals where it is possible to track material streams separately.

However, chemicals are often used in complex combinations, and separate cycles are very often difficult to implement, especially due to prohibitive costs and highly complex stream management, such that the “segregation” model is not always applicable.

Consequently, when it is not possible to differentiate between material streams, other models are applied, which are grouped together under the term “non-segregated” and which entail, for example, taking into account the proportion of a specific stream relative to other streams, without physically separating the streams. One example is the Mass Balance Approach.

The Mass Balance Approach involves accurately tracking the proportion of a category (e.g. “recycled”) relative to a whole in a production system in order to guarantee, on the basis of an auditable account ledger, a proportionate and appropriate allocation of the content of that category in a finished product.

For example, this may be the case of a chemist buying 50% bio-sourced (primary or secondary) alkylamine from a supplier who guarantees, according to the mass or weight balance approach, that in the alkylamine delivered, 50% of the alkylamine has a bio-sourced origin, and de facto 50% is not of bio-sourced origin, and the use by said chemist of this 50% bio-sourced alkylamine with another stream of 0% bio-sourced alkylamine, the two streams not being identifiable at some point during the production process, due to mixing for example. If the chemical compound produced is made from 50% bio-sourced 50 wt % guaranteed alkylamine, and 0% bio-sourced 50 wt % alkylamine, the chemical compound is 25% bio-sourced.

In order to guarantee the stated “bio-sourced” figures, for example, and to encourage the use of recycled raw materials in producing new products, a set of globally shared and standardised rules (ISCC+, ISO 14020) has been developed to reliably manage material streams.

In the context of the invention, the term “recycled” is understood to mean the origin of a chemical compound derived from a method for recycling a material considered as waste, i.e. resulting from one or more transformations carried out using at least one recycling method on at least one material generally considered as waste.

The term “water-soluble polymer” is understood to mean a polymer which gives a clear aqueous solution when dissolved by stirring at 25° C. and with a concentration of 20 g·L⁻¹ in water.

### Method According to the Invention

The present invention relates to a method for obtaining substituted alkyl(meth)acrylamide comprising the reaction between (meth)acrylic acid or one of the esters thereof on the one hand, and a primary or a secondary alkylamine on the other hand, one of the two, preferably both, being at least partially renewable and non-fossil. When referring to the renewable and non-fossil origin, or to one or both, it is understood to mean the renewable and non-fossil origin of the (meth)acrylic acid or one of the esters thereof, and/or the renewable and non-fossil origin of the primary or the secondary alkylamine.

In the whole invention, (meth)acrylic acid or one of the esters thereof is preferentially chosen from formula (1) compounds.

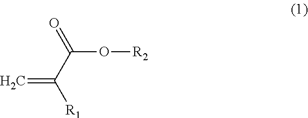

Wherein R₁═H or CH₃, R₂═H, alkyl chain containing 1 to 4 carbon atoms. Preferably R₂═CH₃.

In the whole invention, (primary or secondary) alkylamine is preferentially chosen from formula (2) alkylamines.

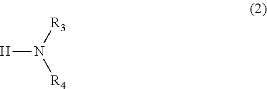

Wherein R₃═H; or alkyl chain comprising 1 to 8 carbon atoms; R₄=alkyl chain containing 1 to 8 carbon atoms, or an alkyl-amine grouping containing 1 to 4 carbon atoms (advantageously dimethylaminopropyl), or alkanol amine (aminoalcohol) containing 1 to 4 carbon atoms; or R₃ and R₄ form a heterocycle of 4 to 6 carbon atoms. In the latter case, the formula (2) alkylamine may be tetrahydro-1,4-oxazine (morpholine). Preferably, (i) R₃ and R₄ are independently of each other a hydrogen atom, a methyl, ethyl, or isopropyl group, or (ii) R₃ and R₄ form a heterocycle of 4 to 6 carbon atoms, preferably morpholine, or (iii) R₃═H and R₄═dimethylaminoprolyl.

R₁ is preferably H in case where (i) R₃═R₄ are independently of each other a methyl, ethyl, or isopropyl group, or isopropyl, or (ii) R₃ and R₄ form a heterocycle to represent tetrahydro-1,4-oxazine (morpholine).

In the case where R₃═H et R₄═dimethylaminoprolyl, R₁ is preferentially CH₃.

In R₂, R₃ and R₄, the aliphatic chains may be linear, branched or cyclic. They are preferably linear.

In the present description, the expressions “between X and Y” and “from X to Y” include the terminals X and Y.

In the whole invention, the bio-sourced carbon content of a compound for which it is specified that it is at least partially renewable and non-fossil, or for which the bio-sourced carbon content is specified, relative to the total carbon weight in said compound, ranges from 5 wt % to 100 wt %, and preferably from 10 wt % to 100 wt %, preferably from 15 wt % to 100 wt %, preferably from 20 wt % to 100 wt %, preferably from 25 wt % to 100 wt %, preferably from 30 wt % to 100 wt %, preferably from 35 wt % to 100 wt %, preferably from 40 wt % to 100 wt %, preferably from 45 wt % to 100 wt %, preferably from 50 wt % to 100 wt %, preferably from 55 wt % to 100 wt %, preferably from 60 wt % to 100 wt %, preferably from 65 wt % to 100 wt %, preferably from 70 wt % to 100 wt %, preferably from 75 wt % to 100 wt %, preferably from 80 wt % to 100 wt %, preferably from 85 wt % to 100 wt %, preferably from 90 wt % to 100 wt %, preferably from 95 wt % to 100 wt %, preferably from 97 wt % to 100 wt %, preferably from 99 wt % to 100 wt %, the bio-sourced carbon content being measured according to ASTM D6866-21 Method B.

In the invention and in the various embodiments described hereinafter, the substituted alkyl(meth)acrylamide obtained according to a method of the invention preferentially has a bio-sourced carbon content ranging between 5 wt % and 100 wt % relative to the total carbon weight in said substituted alkyl(meth)acrylamide, the bio-sourced carbon content being measured according to ASTM D6866-21 Method B.

In the invention and in the various embodiments described hereinafter, the (meth)acrylic acid or one of the esters thereof preferentially has a bio-sourced carbon content ranging between 5 wt % and 100 wt % relative to the total carbon weight in said (meth)acrylic acid or one of the esters thereof, the bio-sourced carbon content being measured according to ASTM D6866-21 Method B.

In the invention and in the various embodiments described hereinafter, the alkylamine has a bio-sourced carbon content ranging between 5 wt % and 100 wt % relative to the total carbon weight in said alkylamine, the bio-sourced carbon content being measured according to ASTM D6866-21 Method B.

In the invention and in the various embodiments described hereinafter, the (meth)acrylic acid or one of the esters thereof is preferentially totally renewable and non-fossil. Preferably, the alkylamine is totally renewable and non-fossil. Preferably, the (meth)acrylic acid or one of the esters thereof, and the alkylamine are totally renewable and non-fossil. Lastly and preferably, the substituted alkyl (meth)acrylamide obtained according to the method of the invention is preferentially totally renewable and non-fossil.

As previously described, the Applicant has particularly observed an improvement of the method where the (meth)acrylic acid used in the method or in preparing the corresponding (meth)acrylic acid ester and used in the method of the invention, is obtained according to a method comprising at least one enzymatic bioconversion step.

Accordingly, in a particularly preferred embodiment, the invention relates to a method for obtaining substituted alkyl(meth)acrylamide comprising the reaction between (meth)acrylic acid or one of the esters thereof on the one hand, and a primary or a secondary alkylamine on the other hand, one of the two, preferentially both, being at least partially renewable and non-fossil, said method comprising a step for obtaining (meth)acrylic acid by a biological method comprising at least a step of enzymatic bioconversion in the presence of at a biocatalyst comprising least one enzyme, with the possibility of said (meth)acrylic acid being converted into a corresponding acrylic acid ester.

In the invention and in the various embodiments described hereinafter, a biological method is understood to mean a method comprising at least one enzymatic bioconversion step in the presence of a biocatalyst comprising at least one enzyme, preferably a method comprising at least two enzymatic bioconversion steps in the presence of a biocatalyst comprising at least one enzyme.

In this preferred embodiment of the invention, the bio-sourced-(meth)acrylic acid is preferentially obtained according to a biological method, either from 3-hydroxypropionitrile that is at least partially renewable and non-fossil, or from (meth)acrylonitrile that is at least partially renewable and non-fossil, said biological method comprising at least one step of enzymatic bioconversion in the presence of a biocatalyst comprising at least one enzyme.

In a first preferred embodiment, the bio-sourced-(meth)acrylic acid is obtained according to a biological method from 3-hydroxypropionitrile that is at least partially renewable and non-fossil.

In a first variant of this first embodiment, the at least partially renewable and non-fossil 3-hydroxypropionitrile is converted into 3-hydroxypropionamide by enzymatic bioconversion in the presence of a biocatalyst comprising at least one nitrile hydratase enzyme, said 3-hydroxypropionamide is then converted into a 3-hydroxypropionic acid salt by enzymatic bioconversion in the presence of a biocatalyst comprising at least one amidase enzyme, said 3-hydroxypropionic acid salt is then converted into 3-hydroxypropionic acid, and lastly, said 3-hydroxypropionic acid is converted into acrylic acid.

In the whole invention, the nitrile hydratase enzyme is preferably synthesized by a microorganism of the type Bacillus, Bacteridium, Micrococcus, Brevibacterium, Corynebacterium, Pseudomonas, Acinetobacter, Xanthobacter, Streptomyces, Rhizobium, Klebsiella, Enterobacter, Erwinia, Aeromonas, Citrobacter, Achromobacter, Agrobacterium, Pseudonocardia, Rhodococcus, Comamonas, Saccharomyces, Dietzia, Clostridium, Lactobacillus, Escherichia, Agrobacterium, Mycobacterium, Methylophilus, Propionibacterium, Actinobacillus, Megasphaera, Aspergillus, Candida, or Fusarium, preferably Rhodococcus rhodochrous, and more preferably Rhodococcus rhodochrous J1.

In the whole invention, the amidase enzyme is preferably synthesized by the following microorganisms: Rhodococcus Erythropolis, Pseudomonas methylotropha, Rhodococcus rhodochrous or Comamonas testosteroni, and more preferably Rhodococcus rhodochrous.

In a second variant of this first embodiment, the at least partially renewable and non-fossil 3-hydroxypropionitrile is converted into a 3-hydroxypropionic acid salt by enzymatic bioconversion in the presence of a biocatalyst comprising at least one nitrilase enzyme, said 3-hydroxypropionic acid salt is then converted into 3-hydroxypropionic acid, and lastly, said 3-hydroxypropionic acid is converted into acrylic acid.

In the whole invention, the nitrilase enzyme is preferably synthesized by a microorganism of the type Bacillus, Bacteridium, Micrococcus, Brevibacterium, Corynebacterium, Pseudomonas, Acinetobacter, Xanthobacter, Streptomyces, Rhizobium, Klebsiella, Enterobacter, Erwinia, Aeromonas, Citrobacter, Achromobacter, Agrobacterium, Pseudonocardia, Rhodococcus, Comamonas, Saccharomyces, Dietzia, Clostridium, Lactobacillus, Escherichia, Agrobacterium, Mycobacterium, Methylophilus, Propionibacterium, Actinobacillus, Megasphaera, Aspergillus, Candida, or Fusarium, preferably Rhodococcus rhodochrous.

In a third variant of this first embodiment, the at least partially renewable and non-fossil 3-hydroxypropionitrile is converted into 3-hydroxypropionamide by enzymatic bioconversion in the presence of a biocatalyst comprising at least one nitrile hydratase enzyme, said 3-hydroxypropionamide is then converted into a 3-hydroxypropionic acid salt by enzymatic bioconversion in the presence of a biocatalyst comprising at least one amidase enzyme, said 3-hydroxypropionic acid salt is then converted into acrylate salt, and lastly, said acrylate salt is converted into acrylic acid.

In a fourth variant of this first embodiment, the at least partially renewable and non-fossil 3-hydroxypropionitrile is converted into a 3-hydroxypropionic acid salt by enzymatic bioconversion in the presence of a biocatalyst comprising at least one nitrilase enzyme, said 3-hydroxypropionic acid salt is then converted into acrylate salt, and lastly, said acrylate salt is converted into acrylic acid.

The (meth)acrylic acid can then be converted into an acrylic acid ester using known methods.

In a second embodiment of this preferred embodiment, the bio-sourced-(meth)acrylic acid is obtained according to a biological method from (meth)acrylonitrile that is at least partially renewable and non-fossil.

In a first variant of the second embodiment, the bio-sourced-(meth)acrylic acid is obtained from (meth)acrylate salt, itself obtained directly from (meth)acrylonitrile that is at least partially renewable and non-fossil by a biological method comprising at least one step of enzymatic hydrolysis of said (meth)acrylonitrile in the presence of a biocatalyst comprising at least one nitrilase enzyme.

In a second variant of the second embodiment, the bio-sourced-(meth)acrylic acid is obtained from (meth)acrylate salt, itself obtained from (meth)acrylamide, itself obtained from (meth)acrylonitrile that is at least partially renewable and non-fossil, by a biological method comprising at least one step of enzymatic hydrolysis of said (meth)acrylamide in the presence of a biocatalyst comprising at least one amidase enzyme in order to obtain the (meth)acrylate salt, and said method comprising at least one step of enzymatic hydrolysis of said (meth)acrylonitrile in the presence of a biocatalyst comprising at least one nitrile hydratase enzyme in order to obtain the (meth)acrylamide

In this second embodiment, the salt obtained is generally an ammonium acrylate or ammonium methacrylate. The method according to the invention further comprises a step wherein the (meth)acrylate salt is converted into (meth)acrylic acid.

The bio-alkylamine may be obtained by reaction between a bio-alcohol and ammonia. For example bio-dimethylamine is obtained from bio-methanol and ammonia. In a first variant, when R₃═R₄═CH₃, the formula (2) bio-product, specifically dimethylamine, is obtained by reaction between biomethanol and ammonia. In a second variant, when R₃═R₄═ethyl, the formula (2) bio-product, specifically diethylamine, is obtained by reaction between bioethanol and ammonia.

In a third variant, when R₃ and R₄ form a heterocycle to represent tetrahydro-1,4-oxazine (morpholine) the formula (2) bio-product is obtained by reaction between bio-diethyleneglycol and ammonia. In a fourth variant, when R₃═H and R₄═dimethylaminoprolyl, the formula (2) bio-product is obtained by reaction between bio-acrylonitrile and bio-dimethylamine, the latter being formed from bio-methanol and ammonia.

The (meth)acrylic acid or one of the esters thereof and/or the alkylamine may be non-segregated, partially segregated, or fully segregated.

Where the (meth)acrylic acid or one of the esters thereof and/or the alkylamine is totally renewable and non-fossil, it may be either:
a) Totally of recycled origin and
a)1) Or totally segregated;
a)2) Or partially segregated;
a)3) Or non-segregated;
b) Or partially of recycled origin and
b)1) Or totally segregated;
b)2) Or partially segregated;
b)3) Or non-segregated;
c) Or totally of non-recycled origin and
c)1) Or totally segregated;
c)2) Or partially segregated;
c)3) Or non-segregated.

In these various embodiments, where the (meth)acrylic acid or one of the esters thereof and/or the alkylamine is partially segregated, the weight ratio between the “segregated” part and the “non-segregated” part is preferably between 99:1 and 10:90, preferably between 99:1 and 30:70, or more preferably between 99:1 and 50:50.

Among these various embodiments, preference is given to the three a) embodiments, the three b) embodiments, and embodiment c)1). Among these embodiments, much greater preference is given to embodiments al)), a)2), b)1), b)2) and c)1). The two most preferred embodiments are a)1) and b)1).

The industrial reality is such that it is not always possible to obtain industrial quantities of (meth)acrylic acid or one of the esters thereof and/or alkylamine that are bio-sourced, totally recycled and/or segregated or highly recycled and segregated. Hence, the above preferences may be more difficult to implement at the moment. From a practical standpoint, embodiments a)3), b)3), and c) are currently implemented more easily and on a larger scale. With techniques evolving quickly towards the circular economy, there is no doubt that the already applicable preferred modes will soon be applicable on a very large scale.

Where the (meth)acrylic acid or one of the esters thereof and/or the alkylamine is partially renewable and non-fossil, a distinction is made between the renewable part (bio-sourced) and the non-bio-sourced part. Obviously, each of these parts can be according to the same embodiments a), b) and c) described hereinabove.

As concerns the bio-sourced part of the bio-sourced (meth)acrylic acid or one of the esters thereof and/or the alkylamine, the same preferences apply as in the case where the compound is fully bio-sourced.

However, as concerns the non-bio-sourced part of the partially bio-sourced compound, it is even more preferable to have as large a recycled component as possible for a circular economy approach.

Hence, in this case, preference is given to embodiments a)1), a)2), b)1), b)2), particularly a)1) and b)1).

In a particular embodiment, (meth)acrylonitrile is obtained using a recycling method.

In this particular embodiment, the (meth)acrylic acid or one of the esters thereof and/or the alkylamine are obtained using a recycling process, such as from polymer depolymerization or by manufacturing from pyrolysis oil, the latter resulting from high-temperature, anaerobic combustion of used plastic waste. Thus, materials considered as waste can be used as a source to produce (meth)acrylic acid or one of the esters thereof and/or alkylamine, which in turn can be used as raw material to manufacture the invention's (alkyl(meth)acrylamide) monomer. Since the monomer according to the invention is derived using a recycling method, the polymer according to the invention hereinafter described can cater to the virtuous circle of the circular economy.

In this particular embodiment, the method according to the invention comprises the following steps:
Recycling at least one material that is at least partially renewable and non-fossil to obtain the (meth)acrylic acid or one of the esters thereof and/or the alkylamine;
Converting the (meth)acrylic acid (or one of the esters thereof) and the alkylamine into substituted alkyl(meth)acrylamide according to one of the method previously described, said method preferably comprising at least one step of enzymatic bioconversion in the presence of a biocatalyst comprising at least one enzyme.

The recycling rate is the weight ratio of the recycled material to the total material.

In this particular embodiment, the part obtained from recycling is preferably totally “segregated”, i.e. is obtained from a separate pipeline and is treated in a separate manner. In an alternative embodiment, it is partially “segregated” and partially “non-segregated”. In this case, the weight ratio between the “segregated” part and the “non-segregated” part is preferably between 99:1 and 10:90, preferably between 99:1 and 30:70, or more preferably between 99:1 and 50:50.

### Monomer According to the Invention

The invention relates to a substituted bio-alkyl(meth)acrylamide obtained by reaction between (meth)acrylic acid or one of the esters thereof on the one hand, and a primary or a secondary alkylamine on the other hand, one of the two, preferentially both, being at least partially renewable and non-fossil. The same embodiments and preferences developed in the “methods” section apply to this section describing the monomer.

The (meth)acrylic acid or one of the esters thereof is preferentially chosen from formula (1) compounds.

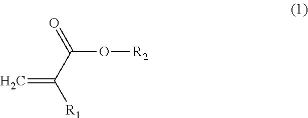

Wherein R₁═H or CH₃, R₂═H, alkyl chain containing 1 to 4 carbon atoms. Preferably, R₂═CH₃.

The alkylamine is preferably selected from the formula (2) alkylamines.

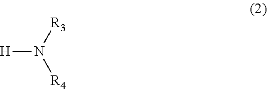

Wherein R₃═H, or alkyl chain comprising 1 to 8 carbon atoms; R₄═alkyl chain containing 1 to 8 carbon atoms, or an alkylamine containing 1 to 4 carbon atoms (dimethylaminopropyl), or alkanol amine containing 1 to 4 carbon atoms; or R₃ and R₄ form a heterocycle of 4 to 6 carbon atoms. In the latter case, the formula (2) alkylamine may be tetrahydro-1,4-oxazine (morpholine). Preferably, (i) R₃ and R₄ are independently of each other a hydrogen atom, a methyl, ethyl, or isopropyl group, or (ii) R₃ and R₄ form a heterocycle of 4 to 6 carbon atoms, preferably morpholine, or (iii) R₃═H and R₄═dimethylaminoprolyl.

R₁ is preferably H in case where (i) R₃═R₄ are independently of each other a methyl, ethyl, or isopropyl group, or isopropyl, or (ii) R₃ and R₄ form a heterocycle to represent tetrahydro-1,4-oxazine (morpholine).

In the case where R₃═H et R₄═dimethylaminoprolyl, R₁ is preferentially CH₃.

In R₂, R₃ and R₄, the aliphatic chains may be linear, branched or cyclic. They are preferably linear.

The substituted bio-alkyl(meth)acrylamide obtained according to a method of the invention preferentially has a bio-sourced carbon content ranging between 5 wt % and 100 wt % relative to the total carbon weight in said substituted bio-alkyl(meth)acrylamide, the bio-sourced carbon content being measured according to ASTM D6866-21 Method B.

The (meth)acrylic acid or one of the esters thereof preferentially has a bio-sourced carbon content ranging between 5 wt % and 100 wt % relative to the total carbon weight in said (meth)acrylic acid, the bio-sourced carbon content being measured according to ASTM D6866-21 Method B.

In the invention and in the various embodiments described hereinafter, the alkylamine preferentially has a bio-sourced carbon content ranging between 5 wt % and 100 wt % relative to the total carbon weight in said alkylamine, the bio-sourced carbon content being measured according to ASTM D6866-21 Method B.

In the invention and in the various embodiments described hereinafter, the (meth)acrylic acid or one of the esters thereof is preferentially totally renewable and non-fossil. Preferably, the alkylamine is totally renewable and non-fossil. Preferably, the (meth)acrylic acid or one of the esters thereof, and the alkylamine are totally renewable and non-fossil. Lastly, the substituted alkyl(meth)acrylamide obtained according to the method of the invention is preferentially totally renewable and non-fossil.

As previously described, the Applicant has particularly observed an improvement of the method where the (meth)acrylic acid used in the method or in preparing the (meth)acrylic acid ester used in the method of the invention, is obtained according to a method comprising at least one enzymatic bioconversion step.

The various embodiments of the substituted alkyl(meth)acrylamide previously described in the “method” section apply to this section describing the monomer.

The (meth)acrylic acid or one of the esters thereof and/or the alkylamine may be non-segregated, partially segregated, or fully segregated. The same embodiments and preferences developed in the “methods” section apply to this section describing the monomer.

In a particular embodiment, the (meth)acrylic acid and or one of the esters thereof and/or the alkylamine may be partially or totally recycled. The same embodiments and preferences developed in the “methods” section apply to this section describing the monomer.

### Polymer According to the Invention

The invention relates to a polymer obtained by polymerization of at least one monomer obtained by the method according to the invention. It also relates to a polymer obtained by polymerization of at least one monomer as previously described. The same embodiments and preferences developed in the “methods” section apply to this section describing the polymer.

The polymer according to the invention offers the advantage of being partially or totally bio-sourced, and where the method of the invention comprises at least one bioconversion step, then the polymer also offers the advantage of being produced according to a biological method known as “soft chemistry”.

The polymer according to the invention is preferably water-soluble or water-swellable. The polymer may also be a superabsorbent.

The polymer according to the invention may be a homopolymer or a copolymer with at least one first monomer obtained by the method according to the invention, or as previously described, and with at least one different additional monomer, the latter advantageously being chosen from at least one nonionic monomer, and/or at least one anionic monomer, and/or at least one cationic monomer, and/or at least one zwitterionic monomer, and/or at least one monomer comprising a hydrophobic grouping.

Thus, the copolymer may comprise at least a second monomer different from the first monomer according to the invention, this second monomer being chosen from nonionic monomers, anionic monomers, cationic monomers, zwitterionic monomers, monomers comprising a hydrophobic grouping, and mixtures thereof.

The nonionic monomer is preferably selected from acrylamide, methacrylamide, N-vinylformamide (NVF), N-vinylacetamide, N-vinylpyridine and N-vinylpyrrolidone (NVP), N-vinyl imidazole, N-vinyl succinimide, acryloyl chloride, glycidyl methacrylate, glyceryl methacrylate, and diacetone acrylamide.

The anionic monomer is preferably chosen from acrylic acid, methacrylic acid, itaconic acid, crotonic acid, maleic acid, fumaric acid, acrylamido undecanoic acid, 3-acrylamido 3-methylbutanoic acid, maleic anhydride, 2-acrylamido-2-methylpropane sulfonic acid (ATBS), vinylsulfonic acid, vinylphosphonic acid, allylsulfonic acid, methallylsulfonic acid, 2-sulfoethylmethacrylate, sulfopropylmethacrylate, sulfopropylacrylate, allylphosphonic acid, styrene sulfonic acid, 2-acrylamido-2-methylpropane disulfonic acid, and the water-soluble salts of these monomers, such as their alkali metal, alkaline earth metal or ammonium salts. It is preferably acrylic acid (and/or a salt thereof), and/or ATBS (and/or a salt thereof).

The cationic monomer is preferably chosen from quaternized dimethylaminoethyl acrylate (ADAME), quaternized dimethylaminoethyl methacrylate (MADAME), dimethyldiallylammonium chloride (DADMAC), acrylamido propyltrimethyl ammonium chloride (APTAC), and methacrylamido propyltrimethyl ammonium chloride (MAPTAC).

The zwitterionic monomer can be a derivative of a vinyl-type unit, particularly acrylamide, acrylic, allylic or maleic, this monomer having an amine or ammonium function (advantageously quaternary) and an acid function of the carboxylic (or carboxylate), sulfonic (or sulfonate) or phosphoric (or phosphate) type.

Monomers having a hydrophobic character can also be used in preparation of the polymer. Preferably, they are chosen from the group composed of esters of (meth)acrylic acid having an alkyl, arylalkyl, propoxylated, ethoxylated or ethoxylated and propoxylated chain; derivatives of (meth)acrylamide having an alkyl, arylalkyl, propoxylated, ethoxylated, ethoxylated and propoxylated chain, or dialkyl; alkyl aryl sulfonates, or of mono- or di-substituted amides of (meth)acrylamide having a propoxylated, ethoxylated, or ethoxylated and propoxylated alkyl, arylalkyl chain; derivatives of (meth)acrylamide having a propoxylated, ethoxylated, ethoxylated and propoxylated alkyl, arylalkyl, or dialkyl chain; alkyl aryl sulfonates

Each of these monomers may also be bio-sourced.

According to the invention, the polymer may have a linear, branched, star, comb, dendritic or block structure. These structures can be obtained by selecting the initiator, the transfer agent, the polymerization technique such as controlled radical polymerization referred to as RAFT (reversible addition-fragmentation chain transfer), NMP (Nitroxide Mediated Polymerization) or ATRP (Atom Transfer Radical Polymerization), incorporation of structural monomers, the concentration . . .

According to the invention, the polymer is advantageously linear and structured. A structured polymer refers to a non-linear polymer with side chains so as to obtain, when this polymer is dissolved in water, a pronounced state of entanglement leading to very substantial low gradient viscosities. The invention's polymer may also be cross-linked.

Additionally, the polymer according to the invention polymer may be structured:
By at least one structuring agent, which may be chosen from the group comprising polyethylenically unsaturated monomers (having at least two unsaturated functions), such as vinyl functions for example, particularly allyl, acrylic and epoxy functions, and one may mention, for example, methylene bis acrylamide (MBA), triallyamine, or tetraallylammonium chloride or 1,2 dihydroxyethylene bis-(N-acrylamide), and/or
By macroinitiators, such as polyperoxides, polyazoids and polytransfer agents, such as polymeric (co)polymers, and polyols, and/or
Functionalized polysaccharides.

The amount of branching/cross-linking agent in the monomer mixture is advantageously less than 4 wt % relative to the monomer content (weight), more advantageously less than 1%, and even more advantageously less than 0.5%. According to a particular embodiment, it may be at least equal to 0.00001 wt % relative to the monomer content.

In a particular embodiment, the polymer according to the invention may be a semi-synthetic and thus semi-natural polymer. In this embodiment, the polymer may be synthesized by copolymerization by total or partial grafting of at least one monomer according to the invention, and at least one natural compound, said natural compound being preferably chosen from starches and their derivatives, polysaccharides and their derivatives, fibers, vegetable gums, animal gums or algal gums, and modified versions thereof. For example, vegetable gums can include guar gum, gum arabic, locust bean gum, gum tragacanth, guanidinium gum, cyanine gum, tara gum, cassia gum, xanthan gum, ghatti gum, karaya gum, gellan gum, cyamopsis tetragonoloba gum, soy gum, or beta-glucan or dammar. The natural compound can also be gelatin, casein, or chitosan. For example, algal gum can include sodium alginate or its acid, agar-agar, or carrageenan.

Polymerization is generally carried out, without this being limiting, by copolymerization or by grafting. The person skilled in the art will be able to refer to current general knowledge in the field of semi-natural polymers.

The invention also relates to a composition comprising at least one polymer according to the invention and at least one natural polymer, said natural polymer being preferably chosen from the previously described natural polymers. The weight ratio between the synthetic polymer and the natural polymer is generally between 90:10 and 10:90. The composition may be in liquid, inverse emulsion or powder form.

In general, the polymer does not require development of a particular polymerization method. Indeed, it can be obtained according to all the polymerization techniques well known to the person skilled in the art. In particular, it can be solution polymerization; gel polymerization; precipitation polymerization; emulsion polymerization (aqueous or inverse); suspension polymerization; reactive extrusion polymerization; water-in-water polymerization; or micellar polymerization.

Polymerization is generally free radical polymerization preferably by inverse emulsion polymerization or gel polymerization. Free radical polymerization includes free radical polymerization using UV, azo, redox or thermal initiators as well as controlled radical polymerization (CRP) techniques or matrix polymerization techniques.

The polymer according to the invention can be modified after it being obtained by polymerization. This is known as post-modification of the polymer. All known post-modifications can be applied to the polymer according to the invention, and the invention also relates to polymers obtained after said post-modifications. Among the possible post-modifications developed hereinafter, mention may be made of post-hydrolysis, post-modification by Mannich reaction, post-modification by Hoffman reaction and post-modification by glyoxalation reaction.

The polymer according to the invention can be obtained by performing a post-hydrolysis reaction on a polymer obtained by polymerization of at least one monomer obtained by the method according to the invention or at least one monomer as previously described in the “Monomer” section. Prior to post-hydrolysis, the polymer comprises acrylamide or methacrylamide monomer units, for example. The polymer may also further comprise monomeric units of N-Vinylformamide. More specifically, post-hydrolysis involves reaction of hydrolyzable functional groups of advantageously non-ionic monomeric units, more advantageously amide or ester functions, with a hydrolysis agent. This hydrolysis agent may be an enzyme, an ion exchange resin, an alkali metal, or a suitable acid compound. Preferably, the hydrolysis agent is a Brønsted base. Where the polymer comprises amide and/or ester monomer units, the post-hydrolysis reaction produces carboxylate groups. Where the polymer comprises vinylformamide monomer units, the post-hydrolysis reaction produces amine groups.

The polymer according to the invention can be obtained by performing a Mannich reaction on a polymer obtained by polymerization of at least one monomer obtained by the method according to the invention or at least one monomer as previously described in the “Monomer” section. More specifically, prior to the Mannich reaction, the polymer advantageously comprises acrylamide and/or methacrylamide monomer units. The Mannich reaction is performed in aqueous solution in the presence of a dialkyl amine and a formaldehyde precursor. More advantageously, the dialkyl amine is dimethylamine and the formaldehyde precursor is formaldehyde itself. After this reaction, the polymer contains tertiary amines.

The polymer according to the invention can be obtained by performing a Hoffman reaction on a polymer obtained by polymerization of at least one monomer obtained by the method according to the invention or at least one monomer as previously described in the “Monomer” section. Prior to the Hoffman reaction, the polymer advantageously comprises acrylamide and/or methacrylamide monomer units. The so-called Hofmann degradation reaction is carried out in aqueous solution in the presence of an alkaline earth and/or alkali hydroxide and an alkaline earth and/or alkali hypohalide.

Discovered by Hofmann at the end of the nineteenth century, this reaction is used to convert an amide function into a primary amine function with one carbon atom less. The detailed reaction mechanism is presented below.

In the presence of a Brønsted base (e.g., soda), a proton is extracted from the amide.

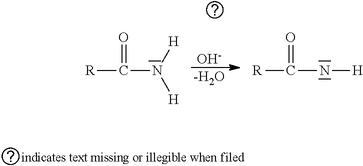

The amidate ion formed then reacts with the active chlorine (Cl₂) of the hypochlorite (e.g. NaClO which is in equilibrium: 2 NaOH+Cl₂⇔NaClO+NaCl+H₂O) to produce an N-chloramide. The Brønsted base (e.g. NaOH) extracts a proton from the chloramide to form an anion. The anion loses a chloride ion to form a nitrene which undergoes isocyanate rearrangement.

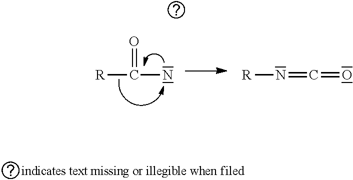

By reaction between the hydroxide ion and the isocyanate, a carbamate is formed.

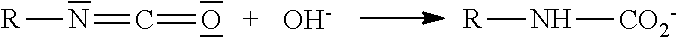

After decarboxylation (removal of CO₂) from the carbamate, a primary amine is obtained.

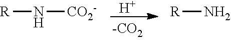

For the conversion of all or part of the amide functions of a (co)polymer comprising an amide group into an amine function, two main factors are involved (expressed in molar ratios). These are:
Alpha=(alkali and/or alkaline earth hypohalide/amide group) and
Beta=(alkali and/or alkaline earth hydroxide/alkali and/or alkaline earth hypohalide).

The polymer according to the invention can also be obtained by carrying out a glyoxalation reaction on a polymer obtained by polymerization of at least one monomer obtained by the method according to the invention or of at least one monomer as previously described in the “Monomer” section, said polymer comprising, with the glyoxalation reaction, at least one monomer unit advantageously of acrylamide or methacrylamide. More specifically, the glyoxalation reaction involves a reaction of at least one aldehyde on the polymer, thus allowing said polymer to be functionalized. Advantageously, the aldehyde may be chosen from the group comprising glyoxal, glutaraldehyde, furan dialdehyde, 2-hydroxyadipaldehyde, succinaldehyde, starch dialdehyde, 2,2 dimethoxyethanal, diepoxy compounds, and combinations thereof. Preferably, the aldehyde compound is glyoxal.

According to the invention, the polymer may be in liquid, gel or solid form when its preparation includes a drying step such as spray drying, drum drying, radiation drying, such as microwave drying, or fluid bed drying.

According to the invention, the water-soluble polymer preferably has a molecular weight between 1000 and 40 million g/mol. The polymer may be a dispersant, in which case its molecular weight is preferably between 1000 and 50,000 g/mol. The polymer may have a higher molecular weight, typically between 1 and 30 million g/mol. The molecular weight is understood as weight average molecular weight. The polymer according to the invention may also be a superabsorbent capable of absorbing from 10 to 500 times its weight in water.

The molecular weight is advantageously determined by the intrinsic viscosity of the (co)polymer. The intrinsic viscosity can be measured by methods known to the person skilled in the art and can be calculated from the reduced viscosity values for different (co)polymer concentrations by a graphical method entailing plotting the reduced viscosity values (y-axis) against the concentration (x-axis) and extrapolating the curve down to zero concentration. The intrinsic viscosity value is plotted on the y-axis or using the least squares method. The molecular weight can then be determined using the Mark-Houwink equation:

$$[ η ] = {KM}^{α}$$

[η] represents the intrinsic viscosity of the (co)polymer determined by the solution viscosity measurement method.
K represents an empirical constant.
M represents the molecular weight of the (co)polymer.
α represents the Mark-Houwink coefficient.
K and α depend on the specific (co)polymer-solvent system.

The co-monomers combined with the monomer according to the invention to obtain the polymer of the invention, are preferably at least partially, or more preferably totally renewable and non-fossil.

Thus, in a preferred embodiment, the invention relates to a polymer comprising:
at least 5 mol %, preferably at least 10 mol %, preferably between 20 mol % and 99 mol %, more preferably between 30 mol % and 90 mol % of a first monomer, said monomer being a monomer according to the invention, and
at least 1 mol %, preferably between 5 mol % and 90 mol %, more preferably between 10 mol % and 80 mol %, of at least one second monomer comprising ethylenic unsaturation, said second monomer being different from the first monomer, and being at least partially renewable and non-fossil.

Thus, in a preferred embodiment, the invention relates to a polymer comprising:
at least 5 mol %, preferably at least 10 mol %, preferably between 20 mol % and 99 mol %, more preferably between 30 mol % and 90 mol % of a first monomer, said monomer being a monomer according to the invention; and
at least 1 mol %, preferably between 5 mol % and 90 mol %, more preferably between 10 mol % and 80 mol %, of at least one second monomer comprising ethylenic unsaturation, said second monomer being different from the first monomer, and being at least partially renewable and non-fossil;
at least 1 mol %, preferably between 5 mol % and 90 mol %, more preferably between 10 mol % and 80 mol % of at least one third monomer comprising an ethylenic unsaturation, said third monomer being different from the first and the second monomers, and being at least partially renewable and non-fossil.

The polymer according to the invention may comprise four or more different monomers.

In a preferred embodiment, the second and the possible other monomers have a bio-sourced carbon content ranging between 5 wt % and 100 wt %, preferably 10 wt % and 100 wt %, relative to the total carbon weight in the related monomer, the bio-sourced carbon content being measured according to ASTM D6866-21 Method B.

In this preferred embodiment, the second and the possible other monomers are preferentially chosen from acrylamide, acrylic acid, an oligomer of acrylic acid, 2-acrylamido-2-methylpropane sulfonic acid (ATBS) and/or one of the salts thereof, N-vinylformamide (NVF), N-vinylpyrrolidone (NVP), dimethyldiallylammonium chloride (DADMAC), quaternized dimethylaminoethyl acrylate (ADAME), quaternized dimethylaminoethyl methacrylate (MADAME).

In the whole invention, it will be understood that the molar percentage of the monomers (excluding any cross-linking agents) of the polymer is equal to 100%.

The (meth)acrylic acid or one of the esters thereof and/or the alkylamine may be non-segregated, partially segregated, or fully segregated. The same embodiments and preferences developed in the “methods” section apply to this section describing the polymer.

In a particular embodiment, the (meth)acrylic acid and or one of the esters thereof and/or the alkylamine may be partially or totally recycled. The same embodiments and preferences developed in the “methods” section apply to this section describing the polymer.

In this particular embodiment, the invention relates to a polymer obtained according to a method comprising the following steps:
Recycling at least one renewable and non-fossil, or a fossil raw material to obtain (meth)acrylic acid or an ester thereof and/or alkylamine;
Reacting said (meth)acrylic acid or an ester thereof and said alkylamine to obtain a substituted alkyl(meth)acrylamide;
Polymerizing said substituted alkyl (meth)acrylamide, and/or optionally another unsaturated monomer to obtain a polymer.

The invention further relates to a polymer as previously described, comprising a bio-sourced carbon content preferentially ranging between 5 wt % and 100 wt % relative to the total carbon weight in said polymer, the bio-sourced carbon content being measured according to ASTM D6866-21 Method B.

The invention also relates to the use of at least one monomer obtained by the method according to the invention in order to synthesize a polymer.

### Using the Polymer According to the Invention

The invention also relates to the use of the polymer according to the invention in the recovery of hydrocarbons (oil and/or gas); in drilling and cementing of wells; in the stimulation of hydrocarbon wells (oil and/or gas), for example hydraulic fracturing, conformation, diversion; in the treatment of water in open, closed or semi-closed circuits; in the treatment of fermentation slurry, treatment of sludge; in paper manufacturing; in construction; in wood processing; in hydraulic composition processing (concrete, cement, mortar and aggregates); in the mining industry; in the formulation of cosmetic products; in the formulation of detergents; in textile manufacturing; in battery component manufacturing; in geothermal energy; in sanitary napkin manufacturing; or in agriculture.

The invention also relates to the use of the polymer according to the invention as a flocculant, coagulant, binding agent, fixing agent, viscosity reducing agent, thickening agent, absorbing agent, friction reducing agent, dewatering agent, draining agent, charge retention agent, dehydrating agent, conditioning agent, stabilizing agent, film forming agent, sizing agent, superplasticizing agent, clay inhibitor or dispersant.

### Method Using the Polymer According to the Invention

The present invention also relates to the various methods described hereinafter, wherein the polymers of the invention are used to improve application performance.

The invention also relates to a method for enhanced oil or gas recovery by sweeping a subterranean formation comprising the following steps:
a. Preparing an injection fluid from a polymer according to the invention with water or brine,
b. Injecting the injection fluid into a subterranean formation,
c. Sweeping the subterranean formation with the injected fluid,
d. Recovering an aqueous mixture of oil and/or gas.

The invention also relates to a method for hydraulic fracturing of subterranean oil and/or gas reservoirs comprising the following steps:
a. Preparing an injection fluid from a polymer according to the invention, with water or brine, and with at least one proppant,
b. Injecting said fluid into the subterranean reservoir and fracturing at least a portion thereof to recover oil and/or gas.

In the methods described hereinabove, the polymer is preferably a high molecular weight polymer (greater than 8 million daltons). It is preferably linear. It is preferably in the form of a powder, an inverse emulsion, a partially dehydrated inverse emulsion, or in the form of a “clear”, i.e. a dispersion of solid polymer particles in an aqueous or oily fluid. The powder form is preferably obtained by gel or spray drying of an inverse emulsion. It also involves a composition comprising an inverse emulsion of a polymer according to the invention and solid particles of a polymer according to the invention.

The invention also relates to a method of stimulation of a subterranean formation comprising the following steps:
a. Preparing an injection fluid from a polymer according to the invention with water or brine,
b. Injecting the injection fluid into a subterranean formation,
c. Partially or totally plugging the subterranean formation with the injected fluid, said plugging being temporary or permanent.

The invention also related a method of drilling and/or cementing a well in a subterranean formation comprising the following steps:
a. Preparing an injection fluid from a polymer according to the invention with water or brine,
b. Injecting said drilling and/or cementing fluid into the subterranean formation via the drill head in at least one step of drilling or cementing a well.

Drilling and cementing a well are two successive steps in creating a well in a subterranean formation. The first step is drilling with the drilling fluid, while the second step is cementing the well with the cementing fluid. The invention also relates to a method of injecting an intermediate fluid (“spacer fluid”) injected between the drilling fluid and the cementing fluid, said intermediate fluid comprising at least one polymer according to the invention. This intermediate fluid prevents contamination between the cementing fluid and the drilling fluid.

When drilling and cementing a well, the polymer according to the invention can be used as a fluid loss additive in well cement compositions in order to reduce fluid loss from the cement compositions to permeable formations or zones into or through which the cement compositions are pumped. In primary cementing, loss of fluid, i.e., water, to permeable formations or subterranean zones can lead to premature gelling of the cement composition, so that bridging the annular space between the permeable formation or zone and the drill string cemented therein prevents the cement composition from being placed along the entire length of the ring.

The invention also relates to a method of inerting clays in hydraulic compositions for construction purposes, said method comprising a step of adding to the hydraulic composition or one of its constituents at least one clay inerting agent, characterized in that the clay inerting agent is a polymer according to the invention.

Clays can absorb water and cause poor performance of building materials. When the polymer of the invention is used as a clay inhibitor, it allows in particular to avoid the clay swelling which may cause cracks thus weakening any building.

The hydraulic composition may be a concrete, cement, mortar or aggregate. The polymer is added to the hydraulic composition or to one of its constituents advantageously at a dosage of 2 to 200 ppm of inerting agent relative to the weight of aggregate.

In this method of inerting clays, clays include, but are not limited to, 2:1 swelling clays (such as smectite), or 1:1 swelling clays (such as kaolin) or 2:1:1 swelling clays (such as chlorite). The term “clay” generally refers to magnesium and/or aluminum silicate, including phyllo silicates with a lamellar structure. However, in the present invention, the term “clay” also includes clays having no such structure, such as amorphous clays.

The invention also relates to a method for manufacturing a sheet of paper, cardboard or the like, whereby, before a sheet is formed, a step is performed entailing adding to a suspension of fibers, at one or more injection points, at least one polymer according to the invention. The polymer may provide dry strength or retention properties or wet strength. It may also improve paper formation, drainage and dewatering capabilities.

The method can be used successfully to manufacture packaging papers and cardboards, coating papers, sanitary and household papers, any type of paper, cardboard or the like.

The post-modified polymers described in the “Polymers” section, in particular the post-modified polymers by Hoffman reaction or by glyoxalation reaction, are particularly advantageous in methods for manufacturing paper, cardboard or the like.

Retention properties are understood to mean the capability to retain the suspended materials of the paper pulp (fibers, fines, fillers (calcium carbonate, titanium oxide), . . . ) on the forming fabric, thus in the fibrous mat that will make up the final sheet. The mode of action of the retention agents is based on the flocculation of these suspended materials in water. Indeed, the flocs formed are more easily retained on the forming sheet.

The retention of fillers involves retaining specifically the fillers (small mineral species with little affinity with cellulose). Substantial improvement of retention of fillers leads to a clarification of white water by retaining the fillers in the sheet and by increasing its grammage. It also gives the possibility to replace part of the fibers (the most expensive species in the composition of paper, cardboard or similar) with fillers (lower costs) in order to reduce manufacturing costs.

As concerns dewatering (or drainage) properties, it is the capacity of the fibrous mat to evacuate or drain the maximum amount of water so that the sheet dries as quickly as possible, in particular during manufacturing of the sheet.

These two properties (retention and drainage) being intricately linked, one depending on the other, the issue is therefore to find the best compromise between retention and drainage. Generally, the person skilled in the art refers to a retention and drainage agent because these are the same types of products used to improve these two properties.

Fibrous suspension is understood to mean thick pulp or diluted pulp which are composed of water and cellulose fibers. The thick stock, with a dry matter concentration of more than 1% or even more than 3%, is located upstream of the fan pump. The thin stock, with a dry mass concentration of generally less than 1%, is located downstream of the fan pump.

The polymer can be added to the thick stock or to the thin stock. It can be added at the level of the fan pump or the headbox. Preferably, the polymer is added before the headbox.

In the method for making paper, cardboard or the like according to the invention, the polymer according to the invention may be used alone or in combination with a secondary retention agent. Preferably, a secondary retention agent selected from organic polymers and/or inorganic microparticles is added to the fiber suspension.

This secondary retention agent added to the fibrous suspension is advantageously chosen from anionic polymers in the broad sense, which can therefore be (without being limiting) linear, branched, cross-linked, hydrophobic, associative and/or inorganic microparticles (such as bentonite, colloidal silica).

The invention also relates to a method for treating a suspension of solid particles in water resulting from mining or oil sands operations, comprising contacting said suspension with at least one polymer according to the invention. Such a method can be carried out in a thickener, which is a holding zone, generally in the form of a tube section of several meters in diameter with a conical bottom wherein the particles can settle. According to a specific embodiment, the aqueous suspension is transported by means of a pipe to a thickener, and the polymer is added to said pipe.

According to another embodiment, the polymer is added to a thickener that already contains the suspension to be treated. In a typical mineral processing operation, the suspensions are often concentrated in a thickener. This results in a higher density sludge that exits the bottom of the thickener, and an aqueous fluid released from the treated suspension (called liquor) that exits by overflow, from the top of the thickener. Generally, the addition of the polymer increases the concentration of the sludge and increases the clarity of the liquor.

According to another embodiment, the polymer is added to the particulate suspension during transport of said suspension to a deposition area. Preferably, the polymer is added in the pipe that conveys said suspension to a deposition zone. It is on this deposition area that the treated suspension is spread in preparation for dewatering and solidification. The deposition areas can be either open, such as an unconfined area of soil, or enclosed, such as a basin, cell.

An example of such treatments during transport of the suspension is spreading the suspension treated with the polymer according to the invention on the soil in preparation for dewatering and solidification and then spreading a second layer of treated suspension on top of the solidified first layer. Another example is the continuous spreading of the suspension treated with the polymer according to the invention in such a way that the treated suspension falls continuously on the suspension previously discharged in the deposition area, thus forming a mass of treated material from which water is extracted.

According to another embodiment, the water-soluble polymer is added to the suspension and a mechanical treatment is performed, such as centrifugation, pressing or filtration.

The water-soluble polymer can be added simultaneously in different stages of the suspension treatment, i.e., for example, in the pipe carrying the suspension to a thickener and in the sludge exiting the thickener which will be conveyed either to a deposition area or to a mechanical treatment device.

The invention also relates to a method for treating municipal or industrial water, comprising the introduction into said water to be treated of at least one polymer according to the invention. Effective water treatment requires the removal of dissolved compounds, and dispersed and suspended solids from the water. Generally, this treatment is enhanced by chemicals such as coagulants and flocculants. These are usually added to the water stream ahead of the separation unit, such as flotation and sedimentation.

The polymers according to the invention can be advantageously used to coagulate or flocculate suspended particles in municipal or industrial wastewater. Generally, they are used in combination with inorganic coagulants such as alum.

They can also be used advantageously to treat the sludge produced from the treatment of this wastewater. Sewage sludge (be it urban or industrial) is the main waste produced by a treatment plant from liquid effluents. Generally, sludge treatment involves dewatering it. This dewatering can be performed by centrifugation, filter press, belt press, electro-dewatering, sludge drying reed beds, solar drying. It is used to decrease sludge water concentration.

In this municipal or industrial water treatment process, the polymer according to the invention is preferably linear or branched. It is preferably in the form of a powder, an inverse emulsion or a partially dehydrated inverse emulsion. The powder form is preferably obtained by gel or spray drying from an inverse emulsion.

The invention also relates to an additive for a cosmetic, dermatological or pharmaceutical composition, said additive comprising at least one polymer according to the invention. The invention also relates to the use of the polymer according to the invention in manufacturing said compositions as a thickening (agent), conditioning (agent), stabilizing (agent), emulsifying (agent), fixing (agent) or film-forming agent. The invention equally relates to cosmetic, dermatological or pharmaceutical compositions comprising at least one polymer according to the invention.

In particular, reference may be made to application FR2979821 on behalf of L'OREAL for the manufacture of such compositions and description of the other ingredients of such compositions. The said compositions may be in the form of a milk, a lotion, a gel, a cream, a gel cream, a soap, a bubble bath, a balm, a shampoo or a conditioner. The use of said compositions for the cosmetic or dermatological treatment of keratinous materials, such as the skin, scalp, eyelashes, eyebrows, nails, hair and/or mucous membranes is also an integral part of the invention. Such use comprises application of the composition to the keratinous materials, possibly followed by rinsing with water.

The invention also relates to an additive for detergent composition, said additive comprising at least one polymer according to the invention. The invention also relates to the use of the polymer according to the invention in manufacturing said compositions as a thickening (agent), conditioning (agent), stabilizing (agent), emulsifying (agent), fixing (agent) or film-forming agent. The invention equally relates to detergent compositions for household or industrial use comprising at least one polymer according to the invention. In particular, reference may be made to the applicant's application WO2016020622 for the manufacture of such compositions and description of the other ingredients of such compositions.

“Detergent compositions for household or industrial use” are understood to mean compositions for cleaning various surfaces, particularly textile fibers, hard surfaces of any kind such as dishes, floors, windows, wood, metal or composite surfaces. Such compositions include, for example, detergents for washing clothes manually or in a washing machine, products for cleaning dishes manually or for dishwashers, detergent products for washing house interiors such as kitchen elements, toilets, furnishings, floors, windows, and other cleaning products for universal use.

The polymer used as an additive, e.g., thickener, for a cosmetic, dermatological, pharmaceutical, or detergent composition is preferably cross-linked. It is preferably in the form of a powder, an inverse emulsion or a partially dehydrated inverse emulsion. The powder form is preferably obtained by spray drying from an inverse emulsion.

The invention equally relates to a thickener for pigment composition used in textile printing, said thickener comprising at least one polymer according to the invention. The invention also relates to a textile fiber sizing agent, said agent comprising at least one polymer according to the invention.

The invention also relates to a process for manufacturing superabsorbent from the monomer according to the invention, a superabsorbent obtained from at least one monomer according to the invention, said superabsorbent to be used for absorbing and retaining water in agricultural applications or for absorbing aqueous liquids in sanitary napkins. For example, the superabsorbent agent is a polymer according to the invention.

The invention also relates to a method for manufacturing sanitary napkins wherein a polymer according to the invention is used, for example as a superabsorbent agent.

The invention also relates to the use of the polymer according to the invention as a battery binder. The invention also relates to a battery binder composition comprising the polymer according to the invention, an electrode material and a solvent. The invention also relates to a method for manufacturing a battery comprising making a gel comprising at least one polymer according to the invention and filling same into said battery. Mention may be made of lithium ion batteries which are used in a variety of products, including medical devices, electric cars, aircraft and, most importantly, consumer products such as laptops, cell phones and cameras.

Generally, lithium ion batteries (LIBs) include an anode, a cathode, and an electrolyte material such as an organic solvent containing a lithium salt. More specifically, the anode and cathode (collectively, the “electrodes”) are formed by mixing an electrode active material (anode or cathode) with a binder and solvent to form a paste or sludge that is then applied and dried onto a current collector, such as aluminum or copper, to form a film on the current collector. The anode and cathode are then stacked and wound before being housed in a pressurized case containing an electrolyte material, all of which together form a lithium-ion battery.

In a lithium battery, the binder plays several important roles in both mechanical and electrochemical performance. Firstly, it helps disperse the other components in the solvent during the manufacturing process (some also act as a thickener), thus allowing for even distribution. Secondly, it holds the various components together, including the active components, any conductive additives, and the current collector, ensuring that all of these parts stay in contact. Through chemical or physical interactions, the binder connects these separate components, holding them together and ensuring the mechanical integrity of the electrode without a material impact on electronic or ionic conductivity. Thirdly, it often serves as an interface between the electrode and the electrolyte. In this role, it can protect the electrode from corrosion or the electrolyte from depletion while facilitating ion transfer across this interface.

Another important point is that the binders must have a certain degree of flexibility so that they do not crack or develop defects. Brittleness can create problems during manufacturing or assembly of the battery.

Given all the roles it plays in an electrode (and in the battery as a whole), choosing a binder is critical in ensuring good battery performance.

The invention also relates to a method for manufacturing sanitary napkins wherein a polymer according to the invention is used, for example as a superabsorbent agent.

As previously described, the circular economy is an economic system devoted to efficiency and sustainability that minimizes waste by optimizing value generated by resources. It relies heavily on a variety of conservation and recycling practices in order to break away from the current more linear “take-make-dispose” approach.

Therefore, with material recycling being a major and growing concern, recycling processes are developing rapidly and enabling the production of materials that can be used to produce new compounds or objects. Recycling materials does not depend on the origin of the material and as long as it can be recycled, it is considered as a technical progress. Although the origin of the material to be recycled may be renewable and non-fossil, it may also be fossil.

Specific objects are described hereinafter.

A specific object relates to a method for obtaining substituted alkyl(meth)acrylamide comprising the reaction between (meth)acrylic acid or one of the esters thereof on the one hand, and a primary or a secondary alkylamine on the other hand, one of the two, preferentially both, being at least partially, preferentially a recycling process of a renewable and non-fossil material, or a fossil material.

Preferentially, the (meth)acrylic acid or one of the esters thereof on the one hand, and the alkylamine on the other hand, are totally “segregated”, i.e., from a separate pipeline and treated separately. In an alternative embodiment, they are partially “segregated” and partially “non-segregated”. In this case, the weight ratio between the “segregated” part and the “non-segregated” part is preferentially between 99:1 and 25:75, preferably between 99:1 and 50:50. In an alternative embodiment, they are totally “non-segregated”.

Another specific object relates to a substituted alkyl(meth)acrylamide obtained by reaction between (meth)acrylic acid or one of the esters thereof on the one hand, and a primary or a secondary alkylamine on the other hand, one of the two, preferentially both, being derived at least partially, preferentially totally from a recycling method of a renewable and non-fossil material, or a fossil material.

Another specific object relates to a polymer obtained by polymerization of at least one substituted alkyl(meth)acrylamide as just previously described.

Another specific object relates to the use of a polymer obtained by polymerization of at least one substituted alkyl(meth)acrylamide as just previously described, in the oil and/or gas recovery, in drilling and cementing of wells; in the stimulation of oil and gas wells (for example hydraulic fracturing, conformation, diversion), in the treatment of water in open, closed or semi-closed circuits, in the treatment of fermentation slurry, treatment of sludge, in paper manufacturing, in construction, in wood processing, in hydraulic composition processing (concrete, cement, mortar and aggregates), in the mining industry, in the formulation of cosmetic products, in the formulation of detergents, in textile manufacturing, in battery component manufacturing; in geothermal energy; or in agriculture.

Another specific object relates to the use of a polymer obtained by polymerization of at least one substituted alkyl(meth)acrylamide as just previously described as a flocculant, coagulant, binding agent, fixing agent, viscosity reducing agent, thickening agent, absorbing agent, friction reducing agent, dewatering agent, draining agent, charge retention agent, dehydrating agent, conditioning agent, stabilizing agent, film forming agent, sizing agent, superplasticizing agent, clay inhibitor or dispersant.

Another specific object relates to a polymer obtained according to a method comprising the following steps:
Recycling at least one renewable and non-fossil, or a fossil raw material to obtain (meth)acrylic acid or an ester thereof and/or alkylamine;
Reacting said (meth)acrylic acid or one of the esters thereof and said alkylamine to obtain a substituted alkyl(meth)acrylamide;
Polymerizing said substituted alkyl (meth)acrylamide, and optionally another unsaturated monomer to obtain a polymer.

The (meth)acrylic acid or one of the esters thereof and/or the alkylamine are preferentially totally “segregated”, i.e., from a separate pipeline and treated separately.

In an alternative embodiment, they are partially “segregated” and partially “non-segregated”. In this case, the weight ratio between the “segregated” part and the “non-segregated” part is preferably between 99:1 and 10:90, preferably between 99:1 and 30:70, or more preferably between 99:1 and 50:50. In an alternative embodiment they are totally “non-segregated”.

### EXAMPLES

In the following examples, it will be a question of synthesizing bio-sourced substituted alkyl (meth)acrylamide comprising the reaction between (meth)acrylic acid or one of its esters on the one hand, and a primary or secondary alkylamine on the other hand, one of the two, preferably both, being at least partly of renewable and non-fossil origin.

These examples best illustrate the advantages of the invention in a clear and non-limiting way.

### Description of the Method of Analysis in Gas Chromatography.

The purity of the various monomers according to the invention is determined by gas chromatography, according to the following conditions:

TABLE 2
gas chromatography analysis method

| | |
|---|---|
| Capillary column | 30 m × 0.25 mm ID, 0.5 μm film |
| | (reference SDB~5MS) |
| Carrier Gas Flow (He) | 1 mL/min |
| Injector | Injection in split ratio 100:1 |
| | Gas saver at 20 mL/min after 2 min |
| | Septum Purge: 3.0 mL/min |
| | Temperature: 325° C. |
| ALS | 1 μL injection |
| Detector: FID (Agilent 8890) | Hydrogen flow: 40 mL/min |
| | Air flow: 400 mL/min |
| | Helium flow: Make Up: 29 mL/min |
| | Temperature: 350° C. |
| Oven temperature | Initial temperature: 30° C. for 5 min |
| | Ramp at 10° C./min up to 325° C. |
| | Final temperature: 325° C. for 2 min |
| | Program duration: 36.5 mins |

Peaks are identified by their retention time. By using external standards and by subtracting the areas of the various impurity peaks, the purity of the monomers of the invention can be calculated.

The retention time of the different products are indicated in table 3 below:

TABLE 3
retention time

| | Retention | | Retention |
|---|---|---|---|
| | time | | time |
| Starting amine | (minutes) | Corresponding monomer | (minutes) |
| Dimethylamine | 2.8 | Dimethylacrylamide | 12.1 |
| Diethylamine | 3.8 | Diethylacrylamide | 14.5 |
| Morpholine | 9 | Acryloyl morpholine | 17.5 |
| Dimethylaminopropylamine | 10 | Dimethylaminopropylacrylamide | 18.5 |

### I. Synthesis of Monomers Described in Table 4

### Example 1: Synthesis of Dimethylacrylamide

A test set is made by adjusting the origin of dimethylamine and its percentage of ¹⁴C

The wt % of ¹⁴C is indicative of the nature of the carbon. The levels of ¹⁴C in the different dimethylamines are measured according to the ASTM D6866-21 standard, method B. This standard makes it possible to characterize the bio-sourced nature of a chemical compound by determining the bio-sourced carbon level of said compound. A “zero pMC” represents the total absence of measurable ¹⁴C in a material, thus indicating a fossil carbon source.

Non-fossil-based dimethylamine can be derived from bio-methanol produced through processing of municipal waste, biomass, by fermentation or carbon dioxide recycling. Alternatively, the amine fraction of the dimethylamine can also be derived from green ammonia.

Methyl acrylate contains 0% ¹⁴C It is of fossil origin.

### Protocol

280 g of methyl acrylate and 100 mg of EMHQ (4-methoxyphenol) are added to a 1000 mL jacketed reactor, equipped with a stirrer and a condenser.

The mixture is heated by a heating unit supplying the reactor jacket until a temperature of 80° C. is reached.

The temperature of the reaction medium is maintained at 50° C., degassing with nitrogen is carried out in order to drive out any oxygen present.

Dimethylamine in gaseous form is added through a bubble tube, for a period of 5 hours. The reaction medium is sampled and then analyzed by proton NMR to ensure that the double bond present on the methyl acrylate has indeed reacted with the dimethylamine through a Michael addition.

8.8 g of sodium methoxide (0.05 molar eq relative to methyl acrylate) are added to the reaction medium.

154 g of gaseous dimethylamine is bubbled in the reactor for a period of 20 hours, in order to promote the amidation reaction.

At the end of the addition, 96% concentrated sulfuric acid in water is added in order to neutralize the sodium methoxide. The sodium sulphate salts are filtered, and the liquid obtained is distilled under slight vacuum at 70° C. to evaporate the light fractions of by-products. The resulting liquid is analyzed by NMR in order to confirm the obtaining of the N-dimethyl-beta-dimethylpropionamide molecule.

The N-dimethyl-beta-dimethylpropionamide is added to a jacketed reactor equipped with a stirrer and a distillation column 10 mm in diameter and 20 cm in height, said column is filled with a packing of the Propack type. The head of the distillation column is connected to a condenser fed with hot water at 500 C, followed by a vacuum trap cooled with liquid nitrogen.

1 ml of 96% concentrated sulfuric acid in water and 1 g of phenothiazine are added to the reaction medium and the latter is heated to a temperature of 200° C.

The entire assembly is also placed under vacuum, at 20 mbar. Thermal decomposition allows dimethylacrylamide and dimethylamine vapor to be obtained. The dimethylacrylamide is collected in the distillate flask of the hot water fed condenser, while the dimethylamine is collected in the vacuum trap.

After 7 hours of reaction, the reaction is stopped, the dimethylacrylamide is collected and weighed then analyzed by gas phase chromatography in order to calculate the yield with respect to the starting methyl acrylate.

The liquid remaining in the reactor is weighed and its fluidity evaluated (Table 4).

In Table 4 below, the dimethylamine is denoted DMA, the methyl acrylate is denoted MA, and the dimethylacrylamide DMAA.

TABLE 4
Synthesis of dimethylacrylamide

| | | | | | | Fluidity of |
|---|---|---|---|---|---|---|
| | | | | | | the liquid |
| | | wt % of ¹⁴C | % | DMA in DMAA | Liquid | remaining in |
| | Origin DMA | of DMA | conversion of MA | obtained (%) | weight (g) | the reactor |
| CE 1 | Fossil | 0 | 78 | 2.5 | 64 | Freeze |
| Inv 1 | Biomethanol | 50 | 82 | 1.5 | 46 | Slightly |
| | (biomass) | | | | | viscous |
| Inv 2 | Biomethanol | 100 | 84 | 1.2 | 41 | Fluid |
| | (biomass) | | | | | |
| Iny 3 | Biomethanol | 50 | 81 | 1.7 | 49 | Slightly |
| | (fermentation) | | | | | viscous |
| Inv 4 | Biomethanol | 100 | 85 | 1.3 | 39 | Fluid |
| | (fermentation) | | | | | |
| Inv 5 | Biomethanol | 50 | 82 | 1.6 | 46 | Slightly |
| | (CO2) | | | | | viscous |
| Inv 6 | Biomethanol | 100 | 85 | 1.3 | 39 | Fluid |
| | (CO2) | | | | | |
| Inv 7 | Biomethanol | 100 | 86 | 0.9 | 36 | Fluid |
| | (biomass) + | | | | | |
| | Green ammonia | | | | | |

(CE = counter-example; Inv = example according to the invention)

The applicant observes that DMA of renewable and non-fossil origin has a higher MA conversion rate than DMA of fossil origin.

### Example 2: Synthesis of Diethylacrylamide

A set of tests is carried out by adjusting the origin of the diethylamine and its percentage of ¹⁴C

Non-fossil-based diethylamine can be derived from bio-ethanol produced through processing of municipal waste, biomass, by fermentation or carbon dioxide recycling. Alternatively, the amine fraction of the diethylamine can also be derived from green ammonia. The level of ¹⁴C in the different diethylamines is measured according to ASTM D6866-21 method B

Methyl acrylate contains 0% ¹⁴C It is of fossil origin.

### Protocol

280 g of methyl acrylate and 100 mg of EMHQ are added to a 1000 mL jacketed reactor, equipped with a stirrer and a condenser. The temperature of the reaction medium is maintained at 50° C., and it is purged with nitrogen in order to drive out the oxygen present therein.

Diethylamine is added for 5 hours. The reaction medium is sampled and analyzed by proton NMR to ensure that the double bond present on the methyl acrylate has indeed reacted with the diethylamine through a Michael addition.

35 g of sodium methoxide (0.2 molar eq relative to methyl acrylate) are added to the reaction medium. 250 g of diethylamide are added to the reactor for a period of 20 hours, in order to promote the amidation reaction.

At the end of the addition, 96% concentrated sulfuric acid in water is added in order to neutralize the sodium methoxide. The sodium sulphate salts are filtered, and the liquid obtained is distilled under slight vacuum at 70° C. to evaporate the light fractions of by-products. The resulting liquid is analyzed by NMR in order to confirm whether the N-dimethyl-beta-dimethylpropionamide molecule has been obtained.

N-diethyl-beta-diethylpropionamide is added to a jacketed reactor equipped with a stirrer and a distillation column of 10 mm diameter and 20 cm height, said column being packed with Propack packing. The head of the distillation column is connected to a condenser fed with hot water at 50° C., followed by a vacuum trap cooled with liquid nitrogen.

1 ml of concentrated sulphuric acid and 1 g of phenothiazine are added to the reaction medium and the latter is heated to 160° C. The whole mixture is also put under a vacuum of 20 mbar. The result of thermal decomposition is diethylacrylamide vapor and diethylamine. The diethylacrylamide is collected in the distillate flask of the condenser supplied with hot water, while the diethylamine is collected in the vacuum trap.

After 20 h, the reaction is stopped, the diethylacrylamide collected is weighed to calculate the yield relative to the starting methyl acrylate, and is analyzed by gas phase chromatography. The liquid remaining in the reactor is weighed and its fluidity evaluated (Table 5).

In the tables below, the diethylamine is denoted DEA, the methyl acrylate is denoted MA, and the diethylacrylamide DEAA.

TABLE 5
Synthesis of diethylacrylamide

| | | | | | | Fluidity of |
|---|---|---|---|---|---|---|
| | | | | | | the liquid |
| | | wt % of ¹⁴C | % | DEA in DEAA | Liquid | remaining in |
| | Origin DEA | of DEA | conversion of MA | obtained (ppm) | weight (g) | the reactor |
| CE 2 | Fossil | 0 | 54 | 3.2 | 113 | Freeze |
| Inv 8 | Bioethanol | 50 | 65 | 2.1 | 56 | Slightly |
| | (biomass) | | | | | viscous |
| Inv 9 | Bioethanol | 100 | 72 | 1.8 | 50 | Fluid |
| | (biomass) | | | | | |
| Inv 10 | Bioethanol | 50 | 64 | 2 | 59 | Slightly |
| | (fermentation) | | | | | viscous |
| Inv 11 | Bioethanol | 100 | 73 | 1.8 | 47 | Fluid |
| | (fermentation) | | | | | |
| Inv 12 | Bioethanol | 50 | 64 | 2 | 56 | Slightly |
| | (CO₂) | | | | | viscous |
| Inv 13 | Bioethanol | 100 | 72 | 1.8 | 49 | Fluid |
| | (CO₂) | | | | | |
| Inv 14 | Bioethanol | 100 | 75 | 1.5 | 42 | Fluid |
| | (biomass) + | | | | | |
| | Green ammonia | | | | | |

(CE = counter-example; Inv = example according to the invention)

### Example 3: Synthesis of Acryloyl Morpholine

A set of tests is carried out by adjusting the origin of the morpholine and its percentage of ¹⁴C

Non-fossil-based morpholine can be derived from bio-ethylene oxide (via bioethanol) produced through processing of municipal waste, biomass, by fermentation or carbon dioxide recycling. Alternatively, the amine fraction of the morpholine can also be derived from green ammonia. The carbon 14 level in the different morpholines is measured according to ASTM D6866-21 method B.

Methyl acrylate contains 0% ¹⁴C It is of fossil origin.

280 g of methyl acrylate and 100 mg of EMHQ are added to a 1 litre jacketed reactor, equipped with a stirrer and a condenser. The temperature of the reaction medium is maintained at 50° C., and is purged with nitrogen to remove the air therein. Morpholine is added, for a period of 5 hours.

The reaction medium is sampled and analyzed by proton NMR to ensure that the double bond present on the methyl acrylate has indeed reacted with the morpholine through a Michael addition.

8.8 g of sodium methoxide (0.05 molar eq relative to methyl acrylate) is added to the reaction medium. 297 g of morpholine is added to the reactor for a period of 10 hours, in order to promote the amidation reaction.

At the end of the addition, 96% sulfuric acid is added to neutralize the sodium methoxide. The sodium sulphate salts are filtered, and the liquid obtained is distilled under slight vacuum at 70° C. to evaporate the light fractions of by-products. The resulting liquid is analyzed by NMR in order to confirm the obtaining of the N-morpholino-beta-morpholinopropionamide molecule.

N-morpholino-beta-morpholinopropionamide is added to a jacketed reactor equipped with a stirrer and a distillation column of 10 mm diameter and 20 cm height, said column being packed with Propack packing. The head of the distillation column is connected to a condenser supplied with hot water at 50° C., followed by a vacuum trap cooled with liquid nitrogen.

1 ml of concentrated sulphuric acid and 1 g of phenothiazine are added to the reaction medium and the latter is heated to 180° C. The whole mixture is also put under a vacuum of 20 mbar. The result of thermal decomposition is acryloyl morpholine vapor and morpholine. The acryloyl morpholine is collected in the distillate flask of the hot water fed condenser, while the morpholine is collected in the vacuum trap.

After 20 hours, the reaction is stopped, the acryloyl morpholine harvested and weighed to calculate the yield relative to the starting methyl acrylate, and is analyzed by gas phase chromatography.

The remaining liquid in the reactor is weighed and its fluidity assessed.

In the tables below, morpholine is denoted MORPH, methyl acrylate is denoted MA, and acryloyl morpholine ACMO.

TABLE 6
Synthesis of acryloyl morpholine

| | | | | | | Fluidity of |
|---|---|---|---|---|---|---|
| | | | | | | the liquid |
| | | wt % of ¹⁴C | % | MORPH in the | Liquid | remaining in |
| | Origin MORPH | of MORPH | conversion of MA | obtained ACMO (%) | weight (g) | the reactor |
| CE 3 | Fossil | 0 | 82 | 2.6 | 60 | Freeze |
| Inv 15 | Bioethanol | 50 | 86 | 1.6 | 46 | Slightly |
| | (biomass) | | | | | viscous |
| Inv 16 | Bioethanol | 100 | 88 | 1.3 | 39 | Fluid |
| | (biomass) | | | | | |
| Inv 17 | Bioethanol | 50 | 85 | 1.8 | 49 | Slightly |
| | (fermentation) | | | | | viscous |
| Inv 18 | Bioethanol | 100 | 89 | 1.4 | 36 | Fluid |
| | (fermentation) | | | | | |
| Inv 19 | Bioethanol | 50 | 86 | 1.7 | 46 | Slightly |
| | (CO₂) | | | | | viscous |
| Inv 20 | Bioethanol | 100 | 89 | 1.4 | 36 | Fluid |
| | (CO₂) | | | | | |
| Iny 21 | Bioethanol | 100 | 90 | 0.9 | 32 | Fluid |
| | (biomass) + | | | | | |
| | green ammonia | | | | | |

(CE = counter-example; Inv = example according to the invention)

### Example 4: Synthesis of Dimethylaminopropylacrylamide

A test set is carried out according to the previous protocol by adjusting the origin of the dimethylaminopropylamine and its percentage of ¹⁴C

Dimethylaminopropylamine of non-fossil origin can be derived from bio-acrylonitrile (via bio-propylene) from the treatment of residues from the paper pulp industry (“tall oil”) or from the treatment of municipal waste, biomass, by fermentation or recycling of carbon dioxide, and dimethylamine of non-fossil origin from bio-methanol produced from the treatment of municipal waste, biomass, by fermentation or recycling of carbon dioxide.

Alternatively the amino moieties of acrylonitrile and dimethylamine can also be derived from green ammonia. The carbon 14 level in the different dimethylaminopropylamines is measured according to ASTM D6866-21 method B.

Methyl acrylate contains 0% ¹⁴C It is of fossil origin.

### Protocol

280 g of methyl acrylate and 100 mg of EMHQ are added to a 1,000 mL jacketed reactor, equipped with a stirrer and a condenser. The temperature of the reaction medium is maintained at 50° C., and is purged with nitrogen to remove the air therein. Dimethylaminopropylamine is added, for a period of 5 hours. The reaction medium is sampled and analyzed by proton NMR to ensure that the double bond present on the methyl acrylate has indeed reacted with the morpholine through a Michael addition.

8.8 g of sodium methoxide (0.05 molar equivalent to methyl acrylate) is added to the reaction medium. 349 g of dimethylaminopropylamine is added to the reactor over a period of 10 hours to promote the amidation reaction.

At the end of the addition, 96% sulfuric acid is added to neutralize the sodium methoxide. The sodium sulphate salts are filtered, and the liquid obtained is distilled under slight vacuum at 70° C. to evaporate the light fractions of by-products. The resulting liquid is analyzed by NMR in order to confirm the obtaining of the N-dimethylaminopropyl-beta-dimethylaminopropylpropionamide molecule.

N-dimethylaminopropyl-beta-dimethylaminopropylpropionamide is added to a jacketed reactor equipped with a stirrer and a distillation column of 10 mm diameter and 20 cm height, said column being packed with Propack packing. The head of the distillation column is connected to a condenser fed with hot water at 50° C., followed by a vacuum trap cooled with liquid nitrogen.

1 ml of concentrated sulphuric acid and Ig of phenothiazine are added to the reaction medium and the latter is heated to 180° C. The whole mixture is also put under a vacuum of 20 mbar. The result of the thermal decomposition is the production of dimethylaminopropylacrylamide vapor and dimethylaminopropylamine. The dimethylaminopropylacrylamide is collected in the distillate flask of the condenser fed with hot water, while the dimethylaminopropylamine is collected in the vacuum trap.

After 20 hours, the reaction is stopped, the dimethylaminopropylacrylamide collected is weighed to calculate the yield relative to the starting methyl acrylate, and is analyzed by gas phase chromatography. The liquid remaining in the reactor is weighed and its fluidity evaluated.

In the tables below, dimethylaminopropylamine is denoted DIMAPA, methyl acrylate is denoted MA, and dimethylaminopropylacrylamide is denoted DMAPAA.

TABLE 7
Synthesis of dimethylaminopropylacrylamide

| | | | | | | Fluidity of |
|---|---|---|---|---|---|---|
| | | | | DIMAPA in | | the liquid |
| | | wt % of ¹⁴C | % | DMAPAA obtained | Liquid | remaining in |
| | Origin DIMAPA | of DIMAPA | conversion of MA | (%) | weight (g) | the reactor |
| CE 4 | Fossil | 0 | 70 | 2.3 | 96 | freeze |
| Inv 22 | Bio I | 60 | 76 | 1.8 | 69 | Slightly |
| | Fossil DMA | | | | | viscous |
| Inv 23 | Fossil ACN | 40 | 75 | 1.9 | 62 | Slightly |
| | Bio-DMA | | | | | viscous |
| Inv 24 | Bio-ACN | 100 | 78 | 1.5 | 74 | Fluid |
| | Bio-DMA | | | | | |
| Inv 25 | Bio-ACN | 100 | 80 | 1.2 | 59 | Fluid |
| | Bio-DMA | | | | | |
| | Green ammonia | | | | | |

(CE = counter-example; Inv = example according to the invention)

### II. Synthesis of Quaternized Monomers

### Example 5: Synthesis of Quaternized Monomers

In a 1000 mL stainless steel reactor, with a pressure-resistant jacket, 300 g of monomers from the invention and counter-example present in table 8 are introduced with stirring. The reactor is closed and pressurized with 1 bar absolute of air.

The reaction medium is heated by a heating unit supplying the reactor jacket until a temperature of 40° C. is reached. Methyl chloride is introduced at a flow rate of 97 g/h. As soon as 10% of the methyl chloride stoichiometry is reached, water is introduced concomitantly at a flow rate of 42 g/h. When all the water has been introduced (i.e. 100 g), the introduction of methyl chloride is stopped and the reactor is returned to atmospheric pressure.

Air is then bubbled in for 30 minutes to de-gas the excess methyl chloride.

An aqueous solution of dimethylaminopropylacrylamide quaternized with methyl chloride is thus obtained. The concentration of this salt is 80% in water.

A set of tests is carried out according to the previous protocol by adjusting the origin of the methyl chloride and its percentage of ¹⁴C (see Table 8).

Non-fossil-based methyl chloride can be derived by processing residues from the pulp and paper industry (“tall oil”) agricultural waste or by processing municipal waste, biomass, by fermentation or carbon dioxide recycling. Alternatively, the chlorine fraction of methyl chloride can also be derived from green chlorine or hydrogen chloride, i.e. manufactured from a renewable energy source.

The level of ¹⁴C in the different products is measured according to the standard ASTM D6866-21 method B.

TABLE 8
¹⁴C level in the monomers (CE = counter-example)

| | | Origin of | wt % of ¹⁴C | wt % of ¹⁴C monomer |
|---|---|---|---|---|
| | Origin of | methyl | of methyl | quaternized with |
| Examples | DMAPAA | chloride | chloride | methyl chloride |
| CE 5 | CE 4 | Fossil | 0 | 33 |
| M1 | Inv 22 | Biomethanol | 100 | 78 |
| M2 | Inv 23 | Biomethanol | 100 | 67 |
| M3 | Inv 24 | Biomethanol | 100 | 100 |
| M4 | Inv 25 | Biomethanol | 100 | 100 |

### III. Polymers According to the Invention

### Example 6: Synthesis and Biodegradability of Polymers P1 to P14

### Synthesis and Biodegradability of Polymers P1 to P6

750 g of water, 200 g of sodium salt of 2-acrylamido-2-methylpropane sulphonic acid, 50 mg of methylene bisacrylamide, 75 mg of sodium hypophosphite and a monomer according to table 9 below are added to a 1000 L reactor, equipped with a jacket, a stirrer and a condenser.

Three hundred g of monomers from the previous monomer are introduced by stirring into a reactor, equipped with a jacket, a stirrer and a condenser. The reactor is closed and pressurized with 1 bar absolute air.

The pH of the reaction medium is adjusted to 7.5 with sodium hydroxide at 20% concentration. The temperature is increased to 55° C., and 400 mg of V-50 is added to the reaction medium. The onset of the polymerization is noted by a rise in temperature, and once the maximum has been reached, the reaction medium is maintained at 70° C. for 60 min.

The viscous liquid obtained is cooled to 20° C. and discharged from the reactor.

The biodegradability (after 28 days) of the polymers obtained is evaluated according to the OECD 302B standard.

TABLE 9
biodegradability of polymers

| | P1 | P2 | P3 | P4 | P5 | P6 | CE6 | CE7 | CE8 |
|---|---|---|---|---|---|---|---|---|---|
| Monomer | Inv 2 | Inv 5 | Inv 8 | Inv 14 | Iny 15 | Inv 21 | CE1 | CE2 | CE3 |
| wt % ¹⁴C of | 100% | 80% | 71% | 100% | 71% | 100% | 60% | 43% | 43% |
| monomer | | | | | | | | | |
| % biodegradability | 28 | 23 | 21 | 29 | 20 | 29 | 10 | 12 | 11 |

(CE = counter-example; Inv = example according to the invention)

The polymers according to the invention have a biodegradability profile that is twice as high as compared to polymers that do not contain bio-based monomers.

### Synthesis and Biodegradability of Polymers P7 to P10, Quaternized

In a 2000 mL beaker, deionized water and quaternized monomers are added (see Table 9).

The resulting solution is cooled to 5-10° C. and transferred to a polymerization reactor. Nitrogen bubbling is carried out for 30 minutes in order to eliminate all traces of dissolved oxygen.

The following are then added to the reactor:
0.45 g of 2,2′-azobisisobutyronitrile,
1.5 mL of an aqueous solution at 2.5 g/L of 2,2′-Azobis[2-(2-imidazolin-2-yl)propane]dihydrochloride,
1.5 mL of an aqueous solution containing 1 g/L of sodium hypophosphite,
1.5 mL of an aqueous solution containing 1 g/L of tert-butyl hydroperoxide,
1.5 mL of an aqueous solution containing 1 g/L of ammonium sulphate and iron (II) hexahydrate (Mohr's salt).

After a few minutes, the nitrogen bubbling is stopped. The polymerization reaction takes place for 4 hours to reach a peak temperature. At the end of this time, the polymer gel obtained is chopped and dried, then again crushed and sieved to obtain a polymer in powder form.

Biodegradability (after 28 days) of the polymers thus obtained is evaluated according to the OECD 302B standard.

TABLE 10
Biodegradability of polymers (CE = counter-example)

| | Polymer | | | | |
|---|---|---|---|---|---|
| | P7 | P8 | P9 | P10 | CE 9 |
| Mass of dimethylaminopropylacrylamide | 252.5 | 252.5 | 252.5 | 252.5 | 252.5 |
| quaternized with methyl chloride (g) | | | | | |

| | Monomer | | | | |
|---|---|---|---|---|---|
| | M1 | M2 | M3 | M4 | CE 5 |
| wt % ¹⁴C of monomer | 78 | 67 | 100 | 100 | 33 |
| Mass of acrylamide (g) | 276 | 276 | 276 | 276 | 276 |
| wt % ¹⁴C of acrylamide | 0 | 0 | 0 | 0 | 0 |
| Mass of water (g) | 522 | 522 | 522 | 522 | 522 |
| % biodegradability | 58 | 55 | 64 | 65 | 23 |

The applicant observes that the polymers P7 to P10, obtained with bio-sourced monomers (containing C¹⁴) are more easily biodegradable than the polymer CE 9.

### Synthesis and Biodegradability of Non-Quaternized Polymers P11 to P14

The protocol previously described is reproduced, except that the monomers used are not quaternized with methyl chloride.

TABLE 11
Biodegradability of polymers (CE = counter-example;
Inv = example according to the invention)

| | Polymer | | | | |
|---|---|---|---|---|---|
| | P11 | P12 | P13 | P14 | CE 10 |
| Mass of | 305 | 305 | 305 | 305 | 305 |
| dimethylaminopropylacrylamide (g) | | | | | |

| | Monomer | | | | |
|---|---|---|---|---|---|
| | Inv 22 | Inv 23 | Inv 24 | Inv 25 | CE 4 |
| wt % ¹⁴C of monomer | 60 | 40 | 100 | 100 | 0 |
| Mass of acrylamide (g) | 276 | 276 | 276 | 276 | 276 |
| wt % ¹⁴C of acrylamide | 0 | 0 | 0 | 0 | 0 |
| Mass of water (g) | 522 | 522 | 522 | 522 | 522 |
| % biodegradability | 52 | 51 | 57 | 58 | 18 |

### IV. Use of the Polymers According to the Invention

### Example 7: Performance of Polymers for Fluid Loss Measurement

Tests have been carried out to show the ability of polymers P1 to P5 to control fluid loss from cement slurries. These tests consist of preparing cement slurries containing the polymers and measuring the filtration of the fluid from the sludge and other properties according to slight variations of the American Petroleum Institute (API) test described in API Specification for Materials and Testing for Well Cements (1982) (Specification 10). The amounts of polymers and water in the mixture are referenced in Table 10, expressed as percentage by weight of API CLASS H dry cement, unless otherwise indicated.

### Protocol:

For each sample tested, 860 g of API Class H cement (Lone Star Industries; Pasadena, Tex.) and 327 g of tap water were mixed at high speed in a Waring Blendor for 35 seconds. Then, 5.95 g (0.5% of total suspension weight and 100% active polymer) of fluid loss control candidate material was added, and the suspension matured at room temperature with stirring for 20 minutes.

To measure fluid loss, the slurry was transferred to a Bariod low pressure filter press (Model 311, NL Baroid/NL Industries, Inc., Houston, Texas). Fluid loss was measured in accordance with API Specification 10, Appendix F (1982) at a differential pressure of 100 PSI and 80° F.

The test results presented in Table 12 demonstrate the significant control of cement fluid loss afforded by the polymers of this invention.

TABLE 12
cement fluid loss

| Polymer | P1 | P2 | P3 | P4 | P5 | P6 | White | CE6 | CE7 | CE8 |
|---|---|---|---|---|---|---|---|---|---|---|
| % polymer weight | 0.5% | 0.5% | 0.5% | 0.5% | 0.5% | 0.5% | 0 | 0.5% | 0.5% | 0.5% |
| Fluid loss (ml) | 42 | 38 | 51 | 49 | 44 | 45 | 1142 | 76 | 83 | 78 |

(CE = counter-example)

### Example 8: Flocculation Test with Monomers According to the Invention

In order to compare the effectiveness of the different polymers P1 to P4 and CE1, comparative flocculation tests were carried out on synthetic water.

The “synthetic” water in the example is prepared from tap water to which 0.015 g/L of humic acid and 2 g/L of kaolin are added.

All the polymers were prepared in dilute solutions, under similar conditions with a dosage of 6 ppm.

The flocculation tests are carried out in a backlit glass column making it possible to measure a sedimentation time between two marks spaced 26 cm apart.

The turbidity of the supernatant is measured. Turbidity refers to the content of suspended matter that clouds the fluid. It is measured using a FLANNA spectrophotometer, which measures the decrease in the intensity of the light ray at an angle of 90°, at a wavelength of 860 nm and expressed in NTU.

The lower the turbidity value, the greater the retention of solid particles.

TABLE 13
flocculation tests

| | Polymer | | | | |
|---|---|---|---|---|---|
| | P7 | P8 | P9 | P10 | CE11 |
| Sedimentation rate (cm/minute) | 24 | 25 | 27 | 27 | 20 |
| Supernatant turbidity (NTU) | 10 | 12 | 11 | 11 | 18 |

Polymers P7 to P10 are better flocculants than polymer CE 11.

TABLE 14
Flocculation tests (CE = counter-example)

| | Polymer | | | | |
|---|---|---|---|---|---|
| | P11 | P12 | P13 | P14 | CE12 |
| Sedimentation rate (cm/minute) | 24 | 25 | 28 | 29 | 20 |
| Supernatant turbidity (NTU) | 9 | 10 | 8 | 7 | 17 |

Polymers P11 to P14 are better flocculants than polymer CE 12.

The quaternization or not of the monomers of the invention does not influence the application efficiency of the final polymers according to the invention. The applicant can, on the other hand, affirm that the bio-sourced nature influences the effectiveness of the application of the polymers.

1. A method for obtaining substituted alkyl(meth)acrylamide comprising the reaction between (meth)acrylic acid or one of the esters thereof on the one hand, and a primary or a secondary alkylamine on the other hand, one of the two, preferably both, being at least partially renewable and non-fossil.

2. The method according to claim 1, characterized in that the substituted alkyl(meth)acrylamide has a bio-sourced carbon content of between 5 wt % and 100 wt % relative to the total carbon weight in said substituted alkyl(meth)acrylamide, the bio-sourced carbon content being measured according to the standard ASTM D6866-21 Method B.

3. The method according to claim 1, characterized in that the (meth)acrylic acid, or one of the esters thereof, has a bio-sourced carbon content ranging between 5 wt % and 100 wt % relative to the total carbon weight in said (meth)acrylic acid, or one of the esters thereof as the case may be, the bio-sourced carbon content being measured according to the standard ASTM D6866-21 Method B.

4. The method according to claim 1, characterized in that the alkylamine has a bio-sourced carbon content of between 5 wt % and 100 wt % relative to the total carbon weight in said alkylamine, the bio-sourced carbon content being measured according to the standard ASTM D6866-21 Method B.

5. The method according to claim 1, characterized in that the (meth)acrylic acid or one of the esters thereof is selected from formula (1) compounds, 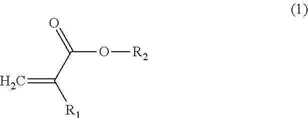
wherein R₁═H or CH₃, R₂═H or an alkyl chain containing 1 to 4 carbon atoms.

6. The method according to claim 1, characterized in that the alkylamine is selected from formula (2) compounds, 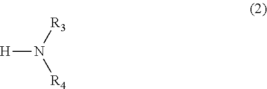
wherein R₃═H, or alkyl chain comprising 1 to 8 carbon atoms; R₄═alkyl chain containing 1 to 8 carbon atoms, or an alkylamine containing 1 to 4 carbon atoms (dimethylaminopropyl), or alkanol amine containing 1 to 4 carbon atoms; or R₃ and R₄ form a heterocycle of 4 to 6 carbon atoms.

7. The method according to claim 1, characterized in that the (meth)acrylic acid or one of the esters thereof and/or the alkylamine are totally renewable and non-fossil.

8. The method according to claim 1, characterized in that said method comprises obtaining (meth)acrylic acid by a biological method comprising at least one step of enzymatic bioconversion in the presence of a biocatalyst comprising at least one enzyme, with the possibility of said (meth)acrylic acid then being converted into a corresponding acrylic acid ester.

9. The method according to claim 1, characterized in that the (meth)acrylic acid is obtained according to a biological method, either from 3-hydroxypropionitrile that is at least partially renewable and non-fossil, or from (meth)acrylonitrile that is at least partially renewable and non-fossil, said biological method comprising at least one step of enzymatic bioconversion in the presence of a biocatalyst comprising at least one enzyme.

10. The method according to claim 1, characterized in that the (meth)acrylic acid or one of the esters thereof and/or the alkylamine are partially or totally segregated.

11. The method according to claim 1, characterized in that the (meth)acrylic acid or one of the esters thereof and/or the alkylamine are derived partially or totally from a recycling process.

12. A substituted bio-sourced alkyl(meth)acrylamide obtained by reaction between (meth)acrylic acid or one of the esters thereof on the one hand, and a primary or a secondary alkylamine on the other hand, one of the two, preferentially both, being at least partially renewable and non-fossil.

13. The substituted bio-sourced alkyl(meth)acrylamide according to claim 12, characterized in that (meth)acrylic acid is obtained by a biological method comprising at least one step of enzymatic bioconversion in the presence of a biocatalyst comprising at least one enzyme, with the possibility of said (meth)acrylic acid then being converted into a corresponding acrylic acid ester.

14. The bio-sourced (meth)acrylamide according to claim 13, characterized in that the (meth)acrylic acid, or one of the esters thereof, has a bio-sourced carbon content ranging between 5 wt % and 100 wt % relative to the total carbon weight in said (meth)acrylic acid or one of the esters thereof as the case may be, and/or in that the alkylamine has a bio-sourced carbon content ranging between 5 wt % and 100 wt % relative to the total carbon weight in said alkylamine, the bio-sourced carbon content being measured according to the standard ASTM D6866-21 Method B.

15. A polymer obtained by polymerization of at least one monomer obtained by the method according to claim 1, wherein the polymer is a copolymer comprising:
at least a first monomer obtained by the method according to claim 1; and
at least a second monomer different from the first monomer, wherein said second monomer is selected from the group consisting of nonionic monomers, anionic monomers, cationic monomers, zwitterionic monomers, monomers comprising a hydrophobic moiety, and mixtures thereof.

16. (canceled)

17. The polymer according to claim 15, characterized in that the polymer is a polymer comprising:
at least 5 mol %, preferably at least 10 mol %, preferentially between 20 mol % and 90 mol %, more preferentially between 30 mol % and 99 mol % of a first monomer, said monomer being a monomer obtained by the method according to claim 1, and
at least 1 mol %, preferentially between 5 mol % and 90 mol %, more preferentially between 10 mol % and 80 mol % of at least one second monomer comprising an ethylenic unsaturation, said second monomer being different from the first monomer, and comprising a bio-sourced carbon content ranging between 5 wt % and 100 wt %, preferably 10 wt % and 100 wt %, relative to the total carbon content in said second monomer, the bio-sourced carbon content being measured according to the standard ASTM D6866-21 Method B.

18. The polymer according to claim 17, characterized in that the at least one second monomer is selected from acrylamide, acrylic acid, an oligomer of acrylic acid, 2-acrylamido-2-methylpropane sulfonic acid (ATBS) and/or one of the salts thereof, N-vinylformamide (NVF), N-vinylpyrrolidone (NVP), dimethyldiallylammonium chloride (DADMAC), quaternized dimethylaminoethyl acrylate (ADAME), quaternized dimethylaminoethyl methacrylate (MADAME).

19. (canceled)

20. (canceled)

21. (canceled)

22. A method for enhanced oil and/or gas recovery by sweeping a subterranean formation, comprising:
a. preparing an injection fluid from a polymer, according to claim 15, with water or brine,
b. injecting the injection fluid into a subterranean formation,
c. sweeping the subterranean formation with the injected fluid,
d. recovering an aqueous mixture of oil and/or gas.

23. A method for hydraulic fracturing of subterranean oil and/or gas reservoirs, comprising:
a. preparing an injection fluid from a polymer, according to claim 15, with water or brine, and with at least one proppant,
b. injecting said fluid into the subterranean reservoir and fracturing at least a portion thereof to recover oil and/or gas.

24. A method of drilling and/or cementing a well in a subterranean formation, comprising:
a. preparing a fluid from a polymer according to claim 15, with water or brine,
b. injecting said drilling and/or cementing fluid into the subterranean formation via the drill head in at least one step of drilling or cementing a well.

25. (canceled)

26. (canceled)

27. (canceled)

28. (canceled)

29. (canceled)
